(19) United States
(12) Patent Application Publication
(10) Pub. No.: US 2026/0317829 A1
(43) Pub. Date: Oct. 15, 2026

(54) METHODS AND MATERIALS FOR ASSESSING AND TREATING CANCERS

(71) Applicant: University of Pittsburgh - of the Commonwealth System of Higher Education, Pittsburgh, PA (US)

(72) Inventors: Udai Shankar Kammula, Pittsburgh, PA (US); Shravan Leonard-Murali, Pittsburgh, PA (US)

(21) Appl. No.: 19/483,723

(22) PCT Filed: Apr. 15, 2024
(86) PCT No.: PCT/US2024/024601

§ 371 Date: Nov. 12, 2025

Related U.S. Application Data
(60) Provisional application No. 63/466,530, filed on May 15, 2023.

(51) Int. Cl. C12Q 1/6886

(52) U.S. Cl. CPC C12Q 1/6886; C12Q 2600/156

(57) ABSTRACT

This document relates to methods and materials involved in assessing a mammal (e.g., a human) having cancer (e.g., metastatic cancer), preparing a treatment for a mammal (e.g., a human) having cancer (e.g., metastatic cancer), and/or treating a mammal (e.g., a human) having cancer (e.g., metastatic cancer). For example, methods and materials for identifying a cancer (e.g., metastatic cancer) as being likely to respond to an adoptive cell therapy (e.g., a tumor infiltrating lymphocyte therapy) are provided.

### CROSS-REFERENCE TO RELATED APPLICATIONS

This application claims the benefit of U.S. Patent Application Ser. No. 63/466,530, filed on May 15, 2023. The disclosure of the prior application is considered part of, and is incorporated by reference in, the disclosure of this application.

### TECHNICAL FIELD

This document relates to methods and materials involved in assessing and/or treating a mammal (e.g., a human) having cancer (e.g., metastatic cancer). For example, methods and materials provided herein can be used to identify a cancer (e.g., metastatic cancer) as being likely to respond to an adoptive cell therapy (ACT; e.g., a tumor infiltrating lymphocyte (TIL) therapy). In another example, methods and materials provided herein can be used to treat a mammal (e.g., a human) having cancer (e.g., metastatic cancer) where the cancer treatment is selected based on whether or not the cancer is likely to be responsive to an ACT (e.g., a TIL therapy).

### BACKGROUND INFORMATION

ACTs have improved cancer outcomes for many patients. For example, a TIL therapy can mediate metastatic tumor regression of multiple solid tumor types that have shown resistance to other cancer treatments such as immune checkpoint inhibitors. However, to determine eligibility for a TIL therapy, patients must be subjected to a surgical harvest of tumor sample, and then wait about three weeks for the tumor sample to be tested for TIL growth and TIL tumor reactivity. This process of testing tumor samples is both time consuming and resource-intensive (see, e.g., Rosenberg et al., Science, 348 (6230): 62-8 (2015); and Chandran et al., Lancet Oncol., 18 (6): 792-802 (2017)).

### SUMMARY

This document provides methods and materials for assessing and/or treating a mammal (e.g., a human) having cancer (e.g., metastatic cancer). In some cases, this document provides methods and materials for identifying whether or not a cancer (e.g., a metastatic cancer) is likely to respond to an ACT such as a TIL therapy (e.g., by detecting the TILScore of the cancer). For example, this document provides methods and materials for detecting the TILScore of a cancer (e.g., a metastatic cancer). In some cases, a TILScore can be detected in a sample (e.g., a tissue sample containing one or more cancer cells) from a mammal having cancer (e.g., metastatic cancer). For example, a sample obtained from a mammal having cancer (e.g., metastatic cancer) can be assessed to determine if the mammal is likely to respond to an ACT (e.g., a TIL therapy) based, at least in part, on the TILScore of the sample.

As demonstrated herein, nucleic acids listed in Table 1 that are differentially expressed in a distinct transcriptomic signature (e.g., a pan-cancer transcriptomic signature) that can be used to calculate a TILScore that can indicate whether or not the mammal (e.g., human) is likely to respond to an ACT (e.g., TIL therapy), regardless of the tumor location and regardless of the tumor origin. For example, a core-like tumor biopsy obtained from a mammal (e.g., a human) having cancer can be used to determine the TILScore of the cancer, thereby determining whether or not the cancer is likely to respond to an ACT (e.g., a TIL therapy). In some cases, a TILScore of a core-like biopsy obtained from a mammal (e.g., a human) having cancer (e.g., metastatic cancer) can be calculated based on a relative rank of at least 30 (e.g., at least 40, at least 50, at least 60, at least 70, at least 80, at least 90, at least 100, at least 125, at least 150, at least 175, at least 200, at least 225, or at least 250) of the nucleic acids listed in Table 1. Also as demonstrated herein, a TILScore of at least 0.15 (e.g., a TILScore of at least 0.15, at least 0.16, at least 0.17, at least 0.18, at least 0.19, at least 0.2, at least 0.3, at least 0.4, at least 0.5, or at least 0.6) can indicate that the cancer is a cancer that is responsive to an ACT such as a TIL therapy. Once a mammal (e.g., a human) is identified as having a cancer that is responsive to an ACT such as a TIL therapy, TILs can be obtained from that mammal and expanded ex vivo to create a culture of tumor reactive TILs that can be administered to that mammal to treat the mammal's cancer.

Having the ability to determine whether or not a cancer (e.g., a metastatic cancer) is likely to respond to an ACT (e.g., a TIL therapy) as described herein (e.g., based, at least in part, on the TILScore of the cancer) allows clinicians to assess this identified patient population in a less-invasive, more efficient, and more accurate manner than current protocols. The ability to determine whether or not a cancer is likely to respond to an ACT (e.g., a TIL therapy) as described herein (e.g., based, at least in part, on the TILScore of the cancer) also allows clinicians to provide a personalized approach in selecting cancer treatments, thereby improving disease-free survival and/or overall survival for this identified patient population. In addition, the ability to determine whether or not a cancer is likely to respond to an ACT (e.g., a TIL therapy) as described herein (e.g., based, at least in part, on the TILScore of the cancer) can minimize subjecting patients to ineffective treatments for this identified patient population. Further, the methods provided herein allow for more frequent sampling that can allow clinicians to monitor tumor evolution (e.g., during a course of treatment).

In general, one aspect of this document features methods for identifying a mammal having cancer likely to respond to an ACT. The methods can include, or consist essentially of, (a) determining that a sample obtained from a mammal and comprising cancer cells has a tumor infiltrating lymphocyte score (TILScore) of at least 0.15, where the TILScore is calculated based on a relative rank of at least 30 nucleic acids listed in Table 1; and (b) classifying the cancer as being likely to respond to the ACT. The mammal can be a human. The ACT can include a TIL therapy. The cancer can include a solid tumor. The at least 30 nucleic acids listed in Table 1 can be at least 50 nucleic acids listed in Table 1. The at least 30 nucleic acids listed in Table 1 can be at least 100 nucleic acids listed in Table 1. The at least 30 nucleic acids listed in Table 1 can include at least 80 percent of nucleic acids listed in Table 1 as having rank #1 to rank #30. The at least 30 nucleic acids listed in Table 1 can include at least 90 percent of nucleic acids listed in Table 1 as having rank #1 to rank #30. The at least 30 nucleic acids listed in Table 1 can include at least 30 nucleic acids listed in Table 1 as having rank #1 to rank #50. The at least 30 nucleic acids listed in Table 1 can include at least 30 nucleic acids listed in Table 1 as having rank #1 to rank #40.

In another aspect, this document features methods for identifying a mammal having cancer unlikely to respond to an ACT. The methods can include, or consist essentially of, (a) determining that a sample obtained from a mammal and comprising cancer cells has a TILScore of less than 0.15, where the TILScore is calculated based on a relative rank of a set of nucleic acids that comprises each of the 30 nucleic acids listed in Table 1 as rank #1 to rank #30; and (b) classifying the cancer as being unlikely to respond to the ACT. The mammal can be a human. The ACT can include a TIL therapy. The cancer can include a solid tumor.

In another aspect, this document features methods for selecting a treatment for a mammal having cancer. The methods can include, or consist essentially of, (a) determining that a sample obtained from a mammal having cancer and comprising cancer cells has a TILScore of at least 0.15, where the TILScore is calculated based on a relative rank of at least 30 nucleic acids listed in Table 1; and (b) selecting an ACT as a treatment of cancer for the mammal. The mammal can be a human. The ACT can include a TIL therapy. The cancer can include a solid tumor. The at least 30 nucleic acids listed in Table 1 can be at least 50 nucleic acids listed in Table 1. The at least 30 nucleic acids listed in Table 1 can be at least 100 nucleic acids listed in Table 1. The at least 30 nucleic acids listed in Table 1 can include at least 80 percent of nucleic acids listed in Table 1 as having rank #1 to rank #30. The at least 30 nucleic acids listed in Table 1 can include at least 90 percent of nucleic acids listed in Table 1 as having rank #1 to rank #30. The at least 30 nucleic acids listed in Table 1 can include at least 30 nucleic acids listed in Table 1 as having rank #1 to rank #50. The at least 30 nucleic acids listed in Table 1 can include at least 30 nucleic acids listed in Table 1 as having rank #1 to rank #40.

In another aspect, this document features methods for selecting a treatment for a mammal having cancer. The methods can include, or consist essentially of, (a) determining that a sample obtained from a mammal having cancer and including cancer cells has a TILScore of less than 0.15, where the TILScore is calculated based on a relative rank of a set of nucleic acids that comprises each of the 30 nucleic acids listed in Table 1 as rank #1 to rank #30; and (b) selecting a cancer treatment other than an ACT for the mammal. The mammal can be a human. The ACT can include a TIL therapy. The cancer can include a solid tumor. The cancer treatment can include radiation therapy. The cancer treatment can include administering an anti-cancer agent selected from the group consisting of a chemotherapy, a targeted therapy, and an angiogenesis inhibitor.

In another aspect, this document features methods for preparing a treatment for a mammal having cancer. The methods can include, or consist essentially of, (a) determining that a sample obtained from the mammal and comprising cancer cells has a TILScore of at least 0.15, where the TILScore is calculated based on a relative rank of at least 30 nucleic acids listed in Table 1; and (b) expanding TILs obtained from the mammal ex vivo to obtain expanded TILs for administration to the mammal. The mammal can be a human. The cancer can include a solid tumor. The at least 30 nucleic acids listed in Table 1 can be at least 50 nucleic acids listed in Table 1. The at least 30 nucleic acids listed in Table 1 can be at least 100 nucleic acids listed in Table 1. The at least 30 nucleic acids listed in Table 1 can include at least 80 percent of nucleic acids listed in Table 1 as having rank #1 to rank #30. The at least 30 nucleic acids listed in Table 1 can include at least 90 percent of nucleic acids listed in Table 1 as having rank #1 to rank #30. The at least 30 nucleic acids listed in Table 1 can include at least 30 nucleic acids listed in Table 1 as having rank #1 to rank #50. The at least 30 nucleic acids listed in Table 1 can include at least 30 nucleic acids listed in Table 1 as having rank #1 to rank #40. The method also can include administering at least a portion of the expanded TILs to the mammal.

In another aspect, this document features methods for preparing a cancer treatment. The methods can include, or consist essentially of, expanding TILs obtained from a mammal identified as having a TILScore of at least 0.15 to form a cell population for administration to the mammal to treat cancer, where the TILScore is calculated based on a relative rank of at least 30 nucleic acids listed in Table 1. The mammal can be a human. The cancer can include a solid tumor. The at least 30 nucleic acids listed in Table 1 can be at least 50 nucleic acids listed in Table 1. The at least 30 nucleic acids listed in Table 1 can be at least 100 nucleic acids listed in Table 1. The at least 30 nucleic acids listed in Table 1 can include at least 80 percent of nucleic acids listed in Table 1 as having rank #1 to rank #30. The at least 30 nucleic acids listed in Table 1 can include at least 90 percent of nucleic acids listed in Table 1 as having rank #1 to rank #30. The at least 30 nucleic acids listed in Table 1 can include at least 30 nucleic acids listed in Table 1 as having rank #1 to rank #50. The at least 30 nucleic acids listed in Table 1 can include at least 30 nucleic acids listed in Table 1 as having rank #1 to rank #40. The method also can include administering at least a portion of the cell population to the mammal.

In another aspect, this document features methods for treating a mammal having cancer. The methods can include, or consist essentially of, (a) determining that a sample obtained from the mammal and comprising cancer cells has a TILScore of at least 0.15, where the TILScore is calculated based on a relative rank of at least 30 nucleic acids listed in Table 1; and (b) administering an ACT to the mammal. The mammal can be a human. The ACT can include a TIL therapy. The cancer can include a solid tumor. The at least 30 nucleic acids listed in Table 1 can be at least 50 nucleic acids listed in Table 1. The at least 30 nucleic acids listed in Table 1 can be at least 100 nucleic acids listed in Table 1. The at least 30 nucleic acids listed in Table 1 can include at least 80 percent of nucleic acids listed in Table 1 as having rank #1 to rank #30. The at least 30 nucleic acids listed in Table 1 can include at least 90 percent of nucleic acids listed in Table 1 as having rank #1 to rank #30. The at least 30 nucleic acids listed in Table 1 can include at least 30 nucleic acids listed in Table 1 as having rank #1 to rank #50. The at least 30 nucleic acids listed in Table 1 can include at least 30 nucleic acids listed in Table 1 as having rank #1 to rank #40.

In another aspect, this document features methods for treating cancer. The methods can include, or consist essentially of, administering an ACT to a mammal identified as having a TILScore of at least 0.15, where the TILScore is determined from a sample obtained from the mammal and comprising cancer cells, and where the TILScore is calculated based on a relative rank of at least 30 nucleic acids listed in Table 1. The mammal can be a human. The ACT can include a TIL therapy. The cancer can include a solid tumor. The at least 30 nucleic acids listed in Table 1 can be at least 50 nucleic acids listed in Table 1. The at least 30 nucleic acids listed in Table 1 can be at least 100 nucleic acids listed in Table 1. The at least 30 nucleic acids listed in Table 1 can include at least 80 percent of nucleic acids listed in Table 1 as having rank #1 to rank #30. The at least 30 nucleic acids listed in Table 1 can include at least 90 percent of nucleic acids listed in Table 1 as having rank #1 to rank #30. The at least 30 nucleic acids listed in Table 1 can include at least 30 nucleic acids listed in Table 1 as having rank #1 to rank #50. The at least 30 nucleic acids listed in Table 1 can include at least 30 nucleic acids listed in Table 1 as having rank #1 to rank #40.

In another aspect, this document features methods for treating a mammal having cancer. The methods can include, or consist essentially of, (a) determining that a sample obtained from the mammal and comprising cancer cells has a TILScore of less than 0.15, where the TILScore is calculated based on a relative rank of a set of nucleic acids that comprises each of the 30 nucleic acids listed in Table 1 as rank #1 to rank #30; and (b) administering a cancer treatment other than an ACT to the mammal. The mammal can be a human. The ACT can include a TIL therapy. The cancer can include a solid tumor. The cancer treatment can include performing surgery. The cancer treatment can include radiation therapy. The cancer treatment can include administering an anti-cancer agent selected from the group consisting of a chemotherapy, a targeted therapy, and an angiogenesis inhibitor.

In another aspect, this document features methods for treating cancer. The methods can include, or consist essentially of, administering a cancer treatment other than an ACT to a mammal identified as having cancer cells having a TILScore of less than 0.15, where the TILScore is calculated based on a relative rank of a set of nucleic acids that comprises each of the 30 nucleic acids listed in Table 1 as rank #1 to rank #30. The mammal can be a human. The ACT can include a TIL therapy. The cancer can include a solid tumor. The cancer treatment can include performing surgery. The cancer treatment can include radiation therapy. The cancer treatment can include administering an anti-cancer agent selected from the group consisting of a chemotherapy, a targeted therapy, and an angiogenesis inhibitor.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. A schematic showing steps and times involved to perform current methods in practice for screening a tumor tissue for TIL reactivity (left side) as compared to steps and times involved to screen a tumor tissue using an exemplary method of one embodiment provided herein (right side).

FIG. 2. A schematic showing TILScore development.

FIG. 3. A pie chart showing the distribution of locations of resected uveal melanoma metastases used to generate the TILScore results provided herein. n=100.

FIG. 4. A heatmap of principal components (PC) correlations with hallmark genesets along with functional annotation of clusters (A, B, C, D).

FIG. 5. Identification of PC2 relationship with Cluster B.

FIG. 6. Gene types in PC2-immune axis gene list.

FIG. 7. Functional annotation of genes in PC2-immune axis gene list.

FIG. 8. Pie charts of tumor histologies in validation and test cohorts.

FIG. 9. TILScore predictive ability in training, validation, and test cohorts.

FIG. 10. TILScore predicted TIL reactivity in the combined pan-cancer cohort.

FIG. 11. Determination of clinically relevant success: futility cutoffs for selected gene sets. The success: futility cutoff was defined as the ability to generate ≥10% tumor reactive TIL cultures from a single metastasis per the gold standard laboratory assay. The graphs display candidate gene set enrichment scores using singscores (x-axis) versus % tumor reactive TIL cultures (y-axis) generated from 194 individual metastases. Success: futility cutoffs were derived from training+validation cohorts of metastases (n=133) and denote the singscore enrichment values equating to 25% probability of yielding significant tumor reactive TIL cultures (≥10). Dashed lines and values represent success: futility cutoffs overlaid onto the total cohort of 194 individual metastases.

FIG. 12. TILScore predicted maximum % change in tumor size from baseline Response Evaluation Criteria in Solid Tumors (RECIST v1.1) response in samples from patients undergoing TIL therapy (n=42).

FIG. 13. Example of TILScore predicting TIL reactivity and response to TIL therapy in a patient with refractory metastatic peritoneal mesothelioma.

FIG. 14. TILScore predicted improved overall survival after ACT in patients undergoing TIL therapy (n=19; TILScore response threshold: 0.235072296; above=greater than, below=less than or equal to).

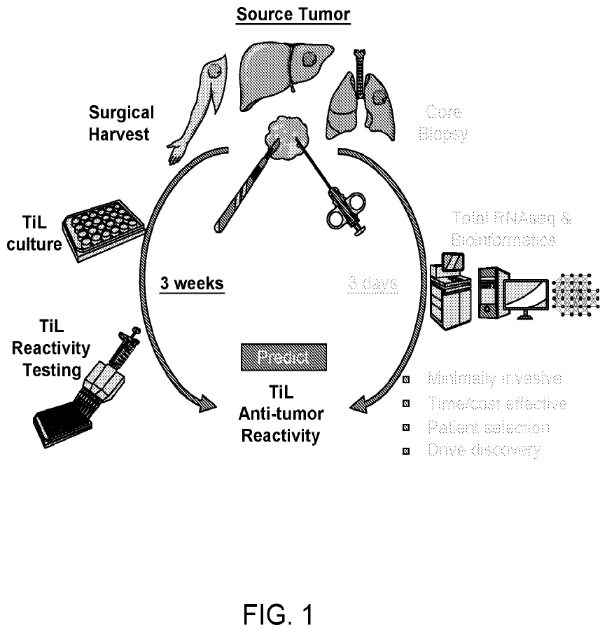

### DETAILED DESCRIPTION

This document provides methods and materials for assessing and/or treating a mammal (e.g., a human) having cancer (e.g., metastatic cancer). For example, this document provides methods and materials for identifying whether or not a cancer (e.g., a metastatic cancer) is likely to respond to an ACT such as a TIL therapy (e.g., by determining the TILScore of the cancer), and, optionally, treating the mammal. In some cases, the methods and materials described herein can be used to predict responsiveness to an ACT (e.g., a TIL therapy). For example, a sample (e.g., a tissue sample containing one or more cancer cells) from a mammal having cancer (e.g., metastatic cancer) can be assessed to determine if the cancer is likely to respond to an ACT (e.g., a TIL therapy) based, at least in part, on the TILScore of the sample.

Once a mammal (e.g., a human) is identified as having a cancer that is responsive to an ACT such as a TIL therapy as described herein, TILs can be obtained from that mammal and expanded ex vivo to create a culture of tumor reactive TILs that can be administered to that mammal to treat the mammal's cancer. For example, a biopsy that includes cancer cells can be obtained from a mammal (e.g., a human) having cancer and can be assessed to determine if the cancer cells have a TILScore that indicates that the mammal's cancer is likely to respond to an ACT (e.g., a TIL therapy) as described herein. If the TILScore of the cancer cells indicates that the mammal's cancer is likely to respond to an ACT (e.g., a TIL therapy), then TILs obtained from that mammal can be expanded ex vivo to create a culture of tumor reactive TILs. Once those tumor reactive TILs are obtained, they can be administered to the mammal to treat the mammal's cancer.

Any appropriate method can be used to expand TILs obtained from a mammal (e.g., a human such as a human identified as having a cancer that is responsive to an ACT such as a TIL therapy as described herein) ex vivo. In some cases, TILs can be expanded ex vivo by culturing the TILs in the presence of one or more polypeptides that can promote the growth and/or differentiation of immune cells. Examples of polypeptides that can promote the growth and/or differentiation of immune cells and that can be used to expand TILs ex vivo include, without limitation, interleukin (IL)-2, IL-7, IL-15, IL-21, and anti-CD3 polypeptides (e.g., anti-CD3 antibodies). In some cases, TILs can be expanded ex vivo by culturing the TILs in the presence of one or more IL-2 polypeptides and in the presence of one or more anti-CD3 antibodies. In some cases, TILs can be expanded ex vivo as described elsewhere (see, e.g., Chandran et al., Lancet Oncol., 18:792-802 (2017) at, for example, page 794).

In some cases, the methods and materials described herein can be used to predict responsiveness to an ACT (e.g., a TIL therapy) in less than about 2 weeks (e.g., less than about 1 week, less than 14 days, less than 13 days, less than 12, days, less than 11 days, less than 10 days, less than 9 days, less than 8 days, less than 7 days, less than 6 days, less than 5 days, less than 4 days, or less than 4 days). For example, a sample (e.g., a tumor tissue sample) from a mammal having cancer (e.g., metastatic cancer) can be assessed to determine if the cancer is likely to respond to an ACT (e.g., a TIL therapy) based, at least in part, on the TILScore of the cancer in from about 2 days to about 2 weeks (e.g., from about 2 days to about 1 week, from about 2 days to about 5 days, from about 5 days to about 2 weeks, from about 1 week to about 2 weeks, or from about 5 days to about 1 week).

A mammal (e.g., a human) having cancer (e.g., metastatic cancer) can be assessed to determine whether the cancer is likely to respond to an ACT (e.g., a TIL therapy) by detecting the TILScore of the cancer. For example, a sample (e.g., a tumor tissue sample) obtained from a mammal having cancer can be assessed to determine if the mammal is likely to respond to an ACT (e.g., a TIL therapy) based, at least in part, on the TILScore of the cancer. For example, a TILScore of a sample (e.g., a tumor tissue sample) obtained from a mammal having cancer can be calculated based on a relative rank of at least 30 (e.g., at least 40, at least 50, at least 60, at least 70, at least 80, at least 90, at least 100, at least 125, at least 150, at least 175, at least 200, at least 225, or at least 250) of the nucleic acids listed in Table 1. A TILScore for a tumor tissue sample is calculated using at least 30 (e.g., at least 40, at least 50, at least 60, at least 70, at least 80, at least 90, at least 100, at least 125, at least 150, at least 175, at least 200, at least 225, or at least 250) of the nucleic acids listed in Table 1 as follows. First, the nucleic acids are ranked based on their transcript abundance in increasing order for the up-set. Second, mean ranks are separately normalized relative to the theoretical minimum and maximum values, centered on zero and then summed to provide the score (e.g., S_{total,i}=S_{up,i}+S_{down,i}), which ranges between −1 and 1. In some cases, a sample with a high score can be interpreted as having a transcriptome that is concordant to the specified signature, and scores reflect the relative mean percentile rank of the TILScore gene set within each sample. The score(S) and normalized score(S) are defined as:

$${S}_{dir , i} = ( \frac{{\sum }_{g} {R}_{dir , i}^{g}}{{N}_{dir , i}} )$$

$${\overline{S}}_{dir , i} = \frac{( {S}_{dir , i} - {S}_{min , i} )}{{S}_{max , i} - {S}_{min , i}}$$

where dir is the gene set direction (e.g., expected up- or down-regulated genes), S_{dir,i} is the score for sample i against the directed gene set, Re^{g}_{dir,i} is the rank of gene g in the directed gene set (e.g., with increasing transcript abundance for expected up-regulated genes and decreasing abundance for expected down-regulated genes), N_{dir,i} is the number of genes in the expected up- or down-regulated gene set that are observed within the data (i.e., signature genes not present within the RNA abundance data are excluded), S̅_{dir,i} is the normalized score for sample i against genes in the signature, and S_{min,i} and S_{max,i} are the theoretical minimum and maximum mean ranks which can be derived from an arithmetic sum (e.g., assuming unique ranks). For a series of n numbers starting at α₁ and with a constant difference d, the sum is calculated as (n/2)(2α₁+(n−1)d). In some cases, setting α₁=1, d=1, n=N_{dir,i} and dividing through by N_{dir,i} is used to obtain the mean:

$${S}_{min , i} = \frac{( {N}_{dir , i} + 1 )}{2}$$

In some cases, the maximum value is obtained by setting α₁=(Nₜₒₜₐₗ−N_{dir}):

$${S}_{max , i} = \frac{( 2 {N}_{total , i} - {N}_{dir , i} + 1 )}{2}$$

where N_{total,i} is the total number of genes in sample i. Example 1 provides an example of calculating a TILScore. See, also, Foroutan et al., BMC Bioinformatics, 19:404 (2018).

Any appropriate mammal having cancer (e.g., metastatic cancer) can be assessed, prepared for treatment, and/or treated as described herein. Examples of mammals that can have cancer and can be assessed, prepared for treatment, and/or treated as described herein include, without limitation, humans, non-human primates (e.g., monkeys), dogs, cats, horses, cows, pigs, sheep, mice, and rats. In some cases, a human having cancer (e.g., metastatic cancer) can be assessed, prepared for treatment, and/or treated as described herein.

When assessing a mammal (e.g., a human) having cancer as described herein, preparing a mammal (e.g., a human) having cancer for treatment as described herein, and/or treating a mammal (e.g., a human) having cancer as described herein, the cancer can be any type of cancer. For example, a cancer assessed and/or treated as described herein can include one or more solid tumors. In some cases, a cancer assessed and/or treated as described herein can be a blood cancer. In some cases, a cancer assessed and/or treated as described herein can be a primary cancer. In some cases, a cancer assessed and/or treated as described herein can be a metastatic cancer. In some cases, a cancer assessed and/or treated as described herein can be a refractory cancer. In some cases, a cancer assessed and/or treated as described herein can be a relapsed cancer. Examples of cancers that can be assessed and/or treated as described herein include, without limitation, uveal melanomas, cutaneous melanomas, liver cancers, lung cancers, breast cancers, lymph cancers, pancreatic cancers (e.g., pancreatic adenocarcinomas), spleen cancers, biliary tract cancers, mesotheliomas (e.g., peritoneal mesotheliomas), Merkel cell carcinomas, sarcomas, gastric cancers (e.g., gastric adenocarcinomas), mucosal melanomas, squamous cell carcinomas, colorectal adenocarcinomas, ovarian carcinomas, gastrointestinal stromal tumors, merkel cell carcinomas, neuroendocrine tumors, urothelial carcinomas, eccrine porocarcinomas, paragangliomas, schwannomas, small bowel adenocarcinomas, and solitary fibrous tumors. In some cases, a mammal (e.g., a human) having cancer and being assessed and/or prepared for treatment (and optionally treated) as described herein, can have multiple (e.g., two or more) different types of cancer. In some cases, a mammal (e.g., a human) having cancer and being assessed and/or prepared for treatment (and optionally treated) as described herein, can have a cancer that has metastasized to one or more different locations.

In some cases, the methods described herein can include identifying a mammal (e.g., a human) as having cancer (e.g., metastatic cancer). Any appropriate method can be used to identify a mammal as having cancer. For example, imaging techniques and biopsy techniques can be used to identify mammals (e.g., humans) as having cancer.

In some cases, a TILScore used to determine whether or not a cancer (e.g., a metastatic cancer) is likely to respond to an ACT (e.g., a TIL therapy) can be calculated based on a relative rank of at least 30 (e.g., at least 40, at least 50, at least 60, at least 70, at least 80, at least 90, at least 100, at least 125, at least 150, at least 175, at least 200, at least 225, or at least 250) of the nucleic acids listed in Table 1. For example, a TILScore of a sample (e.g., a tumor tissue sample) obtained from a mammal having cancer can be calculated based on a relative rank of at least 30 (e.g., at least 40, at least 50, at least 60, at least 70, at least 80, at least 90, at least 100, at least 125, at least 150, at least 175, at least 200, at least 225, or at least 250) of the nucleic acids listed in Table 1 to determine whether or not the cancer is likely to respond to an ACT (e.g., a TIL therapy). In some cases, if a TILScore of cancer cells obtained from a mammal (e.g., a human) having cancer calculated based on a relative rank of at least 30 (e.g., at least 40, at least 50, at least 60, at least 70, at least 80, at least 90, at least 100, at least 125, at least 150, at least 175, at least 200, at least 225, or at least 250) of the nucleic acids listed in Table 1 is at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy). In some cases, if a TILScore of cancer cells obtained from a mammal (e.g., a human) having cancer calculated based on a relative rank of at least 30 of the nucleic acids listed in Table 1 is less than 0.15, then that mammal can be identified as having cancer that is unlikely to respond to an ACT (e.g., a TIL therapy).

In some cases, a TILScore of cancer cells obtained from a mammal (e.g., a human) having cancer calculated based on a relative rank of the 30 nucleic acids listed in Table 1 as having rank #1 to rank #30 that is at least 0.15 can indicate that the mammal has a cancer that is likely to respond to an ACT (e.g., a TIL therapy). In some cases, a TILScore of cancer cells obtained from a mammal (e.g., a human) having cancer calculated based on a relative rank of the 74 nucleic acids listed in Table 1 as having rank #1 to rank #74 that is at least 0.15 can indicate that the mammal has a cancer that is likely to respond to an ACT (e.g., a TIL therapy). Likewise, a TILScore of cancer cells obtained from a mammal (e.g., a human) having cancer calculated based on a relative rank of the 31 nucleic acids listed in Table 1 as having rank #1 to rank #31 that is at least 0.15 can indicate that the mammal has a cancer that is likely to respond to an ACT (e.g., a TIL therapy), a TILScore of cancer cells obtained from a mammal (e.g., a human) having cancer calculated based on a relative rank of the 32 nucleic acids listed in Table 1 as having rank #1 to rank #32 that is at least 0.15 can indicate that the mammal has a cancer that is likely to respond to an ACT (e.g., a TIL therapy), and so forth up to rank #74. In some cases, if a TILScore is not at least 0.15 in the examples of this paragraph, then the mammal can be identified as having cancer that is unlikely to respond to an ACT (e.g., a TIL therapy).

In some cases, a TILScore of cancer cells obtained from a mammal (e.g., a human) having cancer calculated based on a relative rank of the 75 nucleic acids listed in Table 1 as having rank #1 to rank #75 that is at least 0.16 can indicate that the mammal has a cancer that is likely to respond to an ACT (e.g., a TIL therapy). In some cases, a TILScore of cancer cells obtained from a mammal (e.g., a human) having cancer calculated based on a relative rank of the 89 nucleic acids listed in Table 1 as having rank #1 to rank #89 that is at least 0.16 can indicate that the mammal has a cancer that is likely to respond to an ACT (e.g., a TIL therapy). Likewise, a TILScore of cancer cells obtained from a mammal (e.g., a human) having cancer calculated based on a relative rank of the 76 nucleic acids listed in Table 1 as having rank #1 to rank #76 that is at least 0.16 can indicate that the mammal has a cancer that is likely to respond to an ACT (e.g., a TIL therapy), a TILScore of cancer cells obtained from a mammal (e.g., a human) having cancer calculated based on a relative rank of the 77 nucleic acids listed in Table 1 as having rank #1 to rank #77 that is at least 0.16 can indicate that the mammal has a cancer that is likely to respond to an ACT (e.g., a TIL therapy), and so forth up to rank #89. In some cases, if a TILScore is not at least 0.16 in the examples of this paragraph, then the mammal can be identified as having cancer that is unlikely to respond to an ACT (e.g., a TIL therapy).

In some cases, a TILScore of cancer cells obtained from a mammal (e.g., a human) having cancer calculated based on a relative rank of the 90 nucleic acids listed in Table 1 as having rank #1 to rank #90 that is at least 0.17 can indicate that the mammal has a cancer that is likely to respond to an ACT (e.g., a TIL therapy). In some cases, a TILScore of cancer cells obtained from a mammal (e.g., a human) having cancer calculated based on a relative rank of the 95 nucleic acids listed in Table 1 as having rank #1 to rank #95 that is at least 0.17 can indicate that the mammal has a cancer that is likely to respond to an ACT (e.g., a TIL therapy). Likewise, a TILScore of cancer cells obtained from a mammal (e.g., a human) having cancer calculated based on a relative rank of the 91 nucleic acids listed in Table 1 as having rank #1 to rank #91 that is at least 0.17 can indicate that the mammal has a cancer that is likely to respond to an ACT (e.g., a TIL therapy), a TILScore of cancer cells obtained from a mammal (e.g., a human) having cancer calculated based on a relative rank of the 92 nucleic acids listed in Table 1 as having rank #1 to rank #92 that is at least 0.17 can indicate that the mammal has a cancer that is likely to respond to an ACT (e.g., a TIL therapy), and so forth up to rank #95. In some cases, if a TILScore is not at least 0.17 in the examples of this paragraph, then the mammal can be identified as having cancer that is unlikely to respond to an ACT (e.g., a TIL therapy).

In some cases, a TILScore of cancer cells obtained from a mammal (e.g., a human) having cancer calculated based on a relative rank of the 96 nucleic acids listed in Table 1 as having rank #1 to rank #96 that is at least 0.18 can indicate that the mammal has a cancer that is likely to respond to an ACT (e.g., a TIL therapy). In some cases, a TILScore of cancer cells obtained from a mammal (e.g., a human) having cancer calculated based on a relative rank of the 135 nucleic acids listed in Table 1 as having rank #1 to rank #135 that is at least 0.18 can indicate that the mammal has a cancer that is likely to respond to an ACT (e.g., a TIL therapy). Likewise, a TILScore of cancer cells obtained from a mammal (e.g., a human) having cancer calculated based on a relative rank of the 97 nucleic acids listed in Table 1 as having rank #1 to rank #97 that is at least 0.18 can indicate that the mammal has a cancer that is likely to respond to an ACT (e.g., a TIL therapy), a TILScore of cancer cells obtained from a mammal (e.g., a human) having cancer calculated based on a relative rank of the 98 nucleic acids listed in Table 1 as having rank #1 to rank #98 that is at least 0.18 can indicate that the mammal has a cancer that is likely to respond to an ACT (e.g., a TIL therapy), and so forth up to rank #135. In some cases, if a TILScore is not at least 0.18 in the examples of this paragraph, then the mammal can be identified as having cancer that is unlikely to respond to an ACT (e.g., a TIL therapy).

In some cases, a TILScore of cancer cells obtained from a mammal (e.g., a human) having cancer calculated based on a relative rank of the 136 nucleic acids listed in Table 1 as having rank #1 to rank #136 that is at least 0.19 can indicate that the mammal has a cancer that is likely to respond to an ACT (e.g., a TIL therapy). In some cases, a TILScore of cancer cells obtained from a mammal (e.g., a human) having cancer calculated based on a relative rank of the 186 nucleic acids listed in Table 1 as having rank #1 to rank #186 that is at least 0.19 can indicate that the mammal has a cancer that is likely to respond to an ACT (e.g., a TIL therapy). Likewise, a TILScore of cancer cells obtained from a mammal (e.g., a human) having cancer calculated based on a relative rank of the 137 nucleic acids listed in Table 1 as having rank #1 to rank #137 that is at least 0.19 can indicate that the mammal has a cancer that is likely to respond to an ACT (e.g., a TIL therapy), a TILScore of cancer cells obtained from a mammal (e.g., a human) having cancer calculated based on a relative rank of the 138 nucleic acids listed in Table 1 as having rank #1 to rank #138 that is at least 0.19 can indicate that the mammal has a cancer that is likely to respond to an ACT (e.g., a TIL therapy), and so forth up to rank #186. In some cases, if a TILScore is not at least 0.19 in the examples of this paragraph, then the mammal can be identified as having cancer that is unlikely to respond to an ACT (e.g., a TIL therapy).

In some cases, a TILScore of cancer cells obtained from a mammal (e.g., a human) having cancer calculated based on a relative rank of the 187 nucleic acids listed in Table 1 as having rank #1 to rank #187 that is at least 0.2 can indicate that the mammal has a cancer that is likely to respond to an ACT (e.g., a TIL therapy). In some cases, a TILScore of cancer cells obtained from a mammal (e.g., a human) having cancer calculated based on a relative rank of the 265 nucleic acids listed in Table 1 as having rank #1 to rank #265 that is at least 0.2 can indicate that the mammal has a cancer that is likely to respond to an ACT (e.g., a TIL therapy). Likewise, a TILScore of cancer cells obtained from a mammal (e.g., a human) having cancer calculated based on a relative rank of the 188 nucleic acids listed in Table 1 as having rank #1 to rank #188 that is at least 0.2 can indicate that the mammal has a cancer that is likely to respond to an ACT (e.g., a TIL therapy), a TILScore of cancer cells obtained from a mammal (e.g., a human) having cancer calculated based on a relative rank of the 189 nucleic acids listed in Table 1 as having rank #1 to rank #189 that is at least 0.2 can indicate that the mammal has a cancer that is likely to respond to an ACT (e.g., a TIL therapy), and so forth up to rank #265. In some cases, if a TILScore is not at least 0.2 in the examples of this paragraph, then the mammal can be identified as having cancer that is unlikely to respond to an ACT (e.g., a TIL therapy).

In some cases, a TILScore used to determine whether or not a cancer (e.g., a metastatic cancer) is likely to respond to an ACT (e.g., a TIL therapy) can be calculated based on a relative rank of at least 30 percent (e.g., at least 40 percent, at least 50 percent, at least percent, least 70 percent, least 80 percent, or least 90 percent) of the nucleic acids ranked as #1 to #30 in Table 1. For example, TILScore of a sample (e.g., a tumor tissue sample) obtained from a mammal having cancer can be calculated based on a relative rank of at least 30 percent (e.g., at least 40 percent, at least 50 percent, at least 60 percent, least 70 percent, least 80 percent, or least 90 percent) of the nucleic acids ranked as #1 to #30 in Table 1 to determine whether or not the cancer is likely to respond to an ACT (e.g., a TIL therapy). In some cases, if cancer cells obtained from a mammal (e.g., a human) having cancer are determined to have a TILScore of at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy). In some cases, if cancer cells obtained from a mammal (e.g., a human) having cancer are determined to have a TILScore of less than 0.15, then that mammal can be identified as having cancer that is unlikely to respond to an ACT (e.g., a TIL therapy). In some cases, a TILScore used to determine whether or not a cancer (e.g., a metastatic cancer) is likely to respond to an ACT (e.g., a TIL therapy) can be calculated based on a relative rank of each of the nucleic acids ranked as #1 to #30 in Table 1. For example, a TILScore of a sample (e.g., a tumor tissue sample) obtained from a mammal having cancer can be calculated based on a relative rank of each of the nucleic acids ranked as #1 to #30 in Table 1 to determine whether or not the cancer is likely to respond to an ACT (e.g., a TIL therapy). In some cases, if cancer cells obtained from a mammal (e.g., a human) having cancer are determined to have a TILScore of at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy).

In some cases, a TILScore used to determine whether or not a cancer (e.g., a metastatic cancer) is likely to respond to an ACT (e.g., a TIL therapy) can be calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1. For example, a TILScore of a sample (e.g., a tumor tissue sample) obtained from a mammal having cancer can be calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1 to determine whether or not the cancer is likely to respond to an ACT (e.g., a TIL therapy). In some cases, if a TILScore of cancer cells obtained from a mammal (e.g., a human) having cancer calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1 is at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy).

In some cases, a TILScore used to determine whether or not a cancer (e.g., a metastatic cancer) is likely to respond to an ACT (e.g., a TIL therapy) can be calculated based on a relative rank of at least 50 percent (e.g., at least 60 percent, least 70 percent, least 80 percent, or least 90 percent) of the nucleic acids ranked as #1 to #60 in Table 1. For example, a TILScore of a sample (e.g., a tumor tissue sample) obtained from a mammal having cancer can be calculated based on a relative rank of at least 50 percent (e.g., at least 60 percent, least 70 percent, least 80 percent, or least 90 percent) of the nucleic acids ranked as #1 to #60 in Table 1 to determine whether or not the cancer is likely to respond to an ACT (e.g., a TIL therapy). In some cases, if cancer cells obtained from a mammal (e.g., a human) having cancer are determined to have a TILScore of at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy). For example, if a TILScore of cancer cells obtained from a mammal (e.g., a human) having cancer calculated based on a relative rank of at least 40 percent (e.g., at least 50 percent, at least 60 percent, least 70 percent, least 80 percent, or least 90 percent) of the nucleic acids ranked as #1 to #30 in Table 1 and at least 60 percent (e.g., least 70 percent, least 80 percent, or least 90 percent) of the nucleic acids ranked as #31 to #60 in Table 1 is at least than 0.15, then that mammal can be identified as having cancer that is likely to respond to an ACT (e.g., a TIL therapy).

In some cases, a TILScore used to determine whether or not a cancer (e.g., a metastatic cancer) is likely to respond to an ACT (e.g., a TIL therapy) can be calculated based on a relative rank of at least 33.3 percent (e.g., at least 40 percent, at least 50 percent, at least 60 percent, least 70 percent, least 80 percent, or least 90 percent) of the nucleic acids ranked as #1 to #90 in Table 1. For example, a TILScore of a sample (e.g., a tumor tissue sample) obtained from a mammal having cancer can be calculated based on a relative rank of at least 33.3 percent (e.g., at least 40 percent, at least 50 percent, at least 60 percent, least 70 percent, least 80 percent, or least 90 percent) of the nucleic acids ranked as #1 to #90 in Table 1 to determine whether or not the cancer is likely to respond to an ACT (e.g., a TIL therapy). In some cases, if cancer cells obtained from a mammal (e.g., a human) having cancer are determined to have a TILScore of at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy). For example, if a TILScore of cancer cells obtained from a mammal (e.g., a human) having cancer calculated based on a relative rank of at least 30 percent (e.g., at least 40 percent, at least 50 percent, at least 60 percent, least 70 percent, least 80 percent, or least 90 percent) of the nucleic acids ranked as #1 to #30 in Table 1, at least 30 percent (e.g., at least 40 percent, at least 50 percent, at least 60 percent, least 70 percent, least 80 percent, or least 90 percent) of the nucleic acids ranked as #31 to #60 in Table 1, and at least 40 percent (e.g., at least 50 percent, at least 60 percent, least 70 percent, least 80 percent, or least 90 percent) of the nucleic acids ranked as #61 to #90 in Table 1 is at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy).

In some cases, a TILScore used to determine whether or not a cancer (e.g., a metastatic cancer) is likely to respond to an ACT (e.g., a TIL therapy) can be calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #31 to #60 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #61 to #90 in Table 1, and at least 3 (e.g., at least 4, 5, 6, 7, 8, 9, 10, 11, 12, 13, 14, 15, 16, 17, 18, 19, 20, 21, 22, 23, 24, 25, 26, 27, 28, 29, or 30) of the nucleic acids ranked as #91 to #265 in Table 1. For example, a TILScore of a sample (e.g., a tumor tissue sample) obtained from a mammal having cancer can be calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1 and at least 21 (e.g., at least 22, 23, 24, 25, 26, 27, 28, 29, or 30) of the nucleic acids ranked as #31 to #265 in Table 1 to determine whether or not the cancer is likely to respond to an ACT (e.g., a TIL therapy). In some cases, if a TILScore of cancer cells obtained from a mammal (e.g., a human) having cancer calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1 and at least 21 (e.g., at least 22, 23, 24, 25, 26, 27, 28, 29, or 30) of the nucleic acids ranked as #31 to #265 in Table 1 is at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy).

In some cases, a TILScore used to determine whether or not a cancer (e.g., a metastatic cancer) is likely to respond to an ACT (e.g., a TIL therapy) can be calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #31 to #60 in Table 1, and at least 12 (e.g., at least 13, 14, 15, 16, 17, 18, 19, 20, 21, 22, 23, 24, 25, 26, 27, 28, 29, or 30) of the nucleic acids ranked as #61 to #265 in Table 1. For example, a TILScore of a sample (e.g., a tumor tissue sample) obtained from a mammal having cancer can be calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #31 to #60 in Table 1, and at least 12 (e.g., at least 13, 14, 15, 16, 17, 18, 19, 20, 21, 22, 23, 24, 25, 26, 27, 28, 29, or 30) of the nucleic acids ranked as #61 to #265 in Table 1 to determine whether or not the cancer is likely to respond to an ACT (e.g., a TIL therapy). In some cases, if a TILScore of cancer cells obtained from a mammal (e.g., a human) having cancer calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #31 to #60 in Table 1, and at least 12 (e.g., at least 13, 14, 15, 16, 17, 18, 19, 20, 21, 22, 23, 24, 25, 26, 27, 28, 29, or 30) of the nucleic acids ranked as #61 to #265 in Table 1 is at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy).

In some cases, a TILScore used to determine whether or not a cancer (e.g., a metastatic cancer) is likely to respond to an ACT (e.g., a TIL therapy) can be calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #31 to #60 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #61 to #90 in Table 1, and at least 3 (e.g., at least 4, 5, 6, 7, 8, 9, 10, 11, 12, 13, 14, 15, 16, 17, 18, 19, 20, 21, 22, 23, 24, 25, 26, 27, 28, 29, or 30) of the nucleic acids ranked as #91 to #265 in Table 1. For example, a TILScore of a sample (e.g., a tumor tissue sample) obtained from a mammal having cancer can be calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #31 to #60 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #61 to #90 in Table 1, and at least 3 (e.g., at least 4, 5, 6, 7, 8, 9, 10, 11, 12, 13, 14, 15, 16, 17, 18, 19, 20, 21, 22, 23, 24, 25, 26, 27, 28, 29, or 30) of the nucleic acids ranked as #91 to #265 in Table 1 to determine whether or not the cancer is likely to respond to an ACT (e.g., a TIL therapy). In some cases, if a TILScore of cancer cells obtained from a mammal (e.g., a human) having cancer calculated based on a relative rank of at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #1 to #30 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #31 to #60 in Table 1, at least 9 (e.g., at least 10, 11, 12, 13, 14, 15, 16, 17, 18, 19, 20, 21, 22, 23, 24, 25, 26, 27, 28, 29, or all 30) of the nucleic acids ranked as #61 to #90 in Table 1, and at least 3 (e.g., at least 4, 5, 6, 7, 8, 9, 10, 11, 12, 13, 14, 15, 16, 17, 18, 19, 20, 21, 22, 23, 24, 25, 26, 27, 28, 29, or 30) of the nucleic acids ranked as #91 to #265 in Table 1 is at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy).

In some cases, a TILScore used to determine whether or not a cancer (e.g., a metastatic cancer) is likely to respond to an ACT (e.g., a TIL therapy) can be calculated based on a relative rank of at least 30 (e.g., at least 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, or all 265) of the nucleic acids ranked as #1 to #265 in Table 1. For example, a TILScore of a sample (e.g., a tumor tissue sample) obtained from a mammal having cancer can be calculated based on a relative rank of at least 30 (e.g., at least 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, or all 265) of the nucleic acids ranked as #1 to #265 in Table 1 to determine whether or not the cancer is likely to respond to an ACT (e.g., a TIL therapy). In some cases, if a TILScore of cancer cells obtained from a mammal (e.g., a human) having cancer calculated based on a relative rank of at least 30 (e.g., at least 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, or all 265) of the nucleic acids ranked as #1 to #265 in Table 1 is at least 0.15 (e.g., at least 0.15, at least 0.16, at least 0.17, at least 0.18, at least 0.19, at least 0.2, at least 0.3, at least 0.4, at least 0.5, or at least 0.6), then that mammal can be identified as having cancer that is likely to respond to an ACT (e.g., a TIL therapy).

Any appropriate sample from a mammal (e.g., a human) having cancer (e.g., metastatic cancer) can be assessed as described herein (e.g., for a TILScore of the cancer). In some cases, a sample can be a biological sample. In some cases, a sample can contain one or more cancer cells. In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, tissue samples such as tumor tissue samples (e.g., tumor fragments) and tumor single cell suspensions. A sample can be a fresh sample (e.g., a fresh frozen sample). In some cases, one or more biological molecules can be isolated from a sample (e.g., from one or more cancer cells within the sample). For example, nucleic acid can be isolated from a sample and can be assessed as described herein.

Any appropriate method can be used to obtain a sample (e.g., a tumor tissue sample) from a mammal (e.g., a human) having cancer (e.g., metastatic cancer). In some cases, a sample can be obtained using a core biopsy or a core-like biopsy.

In some cases, a sample (e.g., a tumor tissue sample) from a mammal (e.g., a human) having cancer (e.g., metastatic cancer) can be obtained without carrying out surgery.

In some cases, a TILScore of a cancer (e.g., a metastatic cancer) can be used to identify the cancer as being likely to respond to an ACT (e.g., a TIL therapy). For example, a TILScore of at least 0.15 (e.g., at least 0.15, at least 0.16, at least 0.17, at least 0.18, at least 0.19, at least 0.2, at least 0.3, at least 0.4, at least 0.5, or at least 0.6) in a sample (e.g., a tumor tissue sample) obtained from a mammal (e.g., a human) having cancer (e.g., metastatic cancer) calculated based on a relative rank can be used to identify the cancer as being likely to respond to an ACT (e.g., a TIL therapy).

In some cases, a TILScore of a cancer (e.g., a metastatic cancer) can be used to identify the cancer as not being likely to respond to an ACT (e.g., a TIL therapy). For example, a TILScore of less than 0.15 in a sample (e.g., a tumor tissue sample) obtained from a mammal (e.g., a human) having cancer (e.g., metastatic cancer) can be used to identify the cancer as being unlikely to respond to an ACT (e.g., a TIL therapy).

In some cases, a mammal (e.g., a human) having a cancer (e.g., a metastatic cancer) that is identified as being likely to respond to an ACT (e.g., a TIL therapy) as described herein (e.g., based, at least in part, on a TILScore of the cancer) can be selected to receive an ACT (e.g., a TIL therapy) to treat the cancer. For example, a mammal having a cancer (e.g., a metastatic cancer) and identified as having a TILScore of at least 0.15 (e.g., at least 0.15, at least 0.16, at least 0.17, at least 0.18, at least 0.19, at least 0.2, at least 0.3, at least 0.4, at least 0.5, or at least 0.6) can be selected to receive an ACT (e.g., a TIL therapy).

In some cases, a mammal (e.g., a human) having a cancer (e.g., a metastatic cancer) that is identified as not being likely to respond to an ACT (e.g., a TIL therapy) as described herein (e.g., based, at least in part, on a TILScore of the cancer) can be selected to receive an alternative cancer treatment (e.g., one or more cancer treatments that are not an ACT) to treat the cancer. For example, a mammal having a cancer (e.g., a metastatic cancer) that is identified as having a TILScore of less than 0.15 can be selected to receive an alternative cancer treatment (e.g., one or more cancer treatments that are not an ACT).

This document also provides methods and materials for treating a mammal (e.g., a human) having cancer (e.g., metastatic cancer). In some cases, a mammal (e.g., a human) having cancer and assessed as described herein (e.g., to determine whether or not the cancer is likely to respond to an ACT such as a TIL therapy based, at least in part, on a TILScore of the cancer) can be administered or instructed to self-administer one or more (e.g., one, two, three, four, five, or more) cancer treatments, where the one or more cancer treatments are effective to treat the cancer within the mammal. For example, a mammal having cancer can be administered or instructed to self-administer one or more cancer treatments selected based, at least in part, on whether or not the cancer is likely to respond to an ACT such as a TIL therapy (e.g., based, at least in part, on a TILScore of the cancer).

When treating a mammal (e.g., a human) having a cancer (e.g., a metastatic cancer) that is identified as being likely to respond to an ACT (e.g., a TIL therapy) as described herein (e.g., based, at least in part, on a TILScore of the cancer), the mammal can be administered or instructed to self-administer an ACT. For example, a mammal having a cancer (e.g., a metastatic cancer) identified as having a TILScore of at least 0.15 (e.g., at least 0.15, at least 0.16, at least 0.17, at least 0.18, at least 0.19, at least 0.2, at least 0.3, at least 0.4, at least 0.5, or at least 0.6) can be administered or instructed to self-administer one or more ACTs. Examples of ACTs that can be administered to a mammal (e.g., a human) having a cancer (e.g., a metastatic cancer) identified has being likely to respond to an ACT as described herein include, without limitation, TIL therapies.

When treating a mammal (e.g., a human) having a cancer (e.g., a metastatic cancer) that is identified as not being likely to respond to an ACT (e.g., a TIL therapy) as described herein (e.g., based, at least in part, on a TILScore of the cancer), the mammal can be administered or instructed to self-administer one or more (e.g., one, two, three, four, five, or more) alternative cancer treatments (e.g., one or more cancer treatments that are not an ACT such as a TIL therapy). For example, a mammal having a cancer (e.g., a metastatic cancer) identified as having a TILScore of less than 0.15 can be administered or instructed to self-administer one or more alternative cancer treatments that are not an ACT (e.g., a TIL therapy). Examples of alternative cancer treatments that are not an ACT (e.g., a TIL therapy) include, without limitation, performing surgery, performing radiation therapies, and administering one or more anti-cancer agents (e.g., chemotherapies, administering targeted therapies (e.g., monoclonal antibody therapies), and administering angiogenesis inhibitors).

In some cases, when treating a mammal (e.g., a human) having cancer (e.g., metastatic cancer) as described herein, the treatment can be effective to treat the cancer. For example, the number of cancer cells present within a mammal can be reduced using the methods and materials described herein. In some cases, the size (e.g., volume) of one or more tumors present within a mammal can be reduced using the methods and materials described herein. For example, the methods and materials described herein can be used to reduce the size of one or more tumors present within a mammal having cancer (e.g., metastatic cancer) by, for example, 10, 20, 30, 40, 50, 60, 70, 80, 90, 95, or more percent. In some cases, the methods and materials described herein can be used to treat cancer in a manner such that the size (e.g., volume) of one or more tumors present within a mammal does not increase.

In some cases, when treating a mammal (e.g., a human) having cancer (e.g., metastatic cancer) as described herein, the treatment can be effective to improve survival of the mammal. For example, the methods and materials described herein can be used to improve disease-free survival (e.g., relapse-free survival). For example, the methods and materials described herein can be used to improve progression-free survival. For example, the methods and materials described herein can be used to improve the survival of a mammal having cancer (e.g., metastatic cancer) by, for example, 10, 20, 30, 40, 50, 60, 70, 80, 90, 95, or more percent. For example, the methods and materials described herein can be used to improve the survival of a mammal having cancer (e.g., metastatic cancer) by, for example, at least 6 months (e.g., about 6 months, about 8 months, about 10 months, about 1 year, about 1.5 years, about 2 years, about 2.5 years, or about 3 years).

The invention will be further described in the following examples, which do not limit the scope of the invention described in the claims.

### EXAMPLES

### Example 1: Transcriptomic-Guided Tumor Infiltrating Lymphocyte (TIL) Therapy

This Example describes the identification of a pan-cancer transcriptomic signature (TILScore) that can be used to predict whether or not a tumor is likely to respond to an adoptive cell therapy (e.g., a TIL therapy).

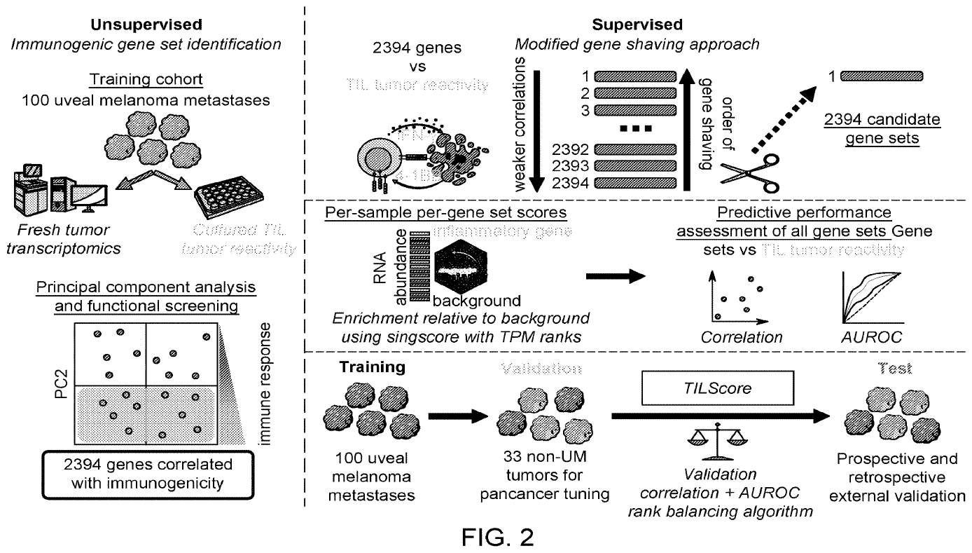

The methodology used to identify nucleic acids and generate a TILScore is shown generally in FIG. 2.

### Development of TILScore

### Discovery Cohort

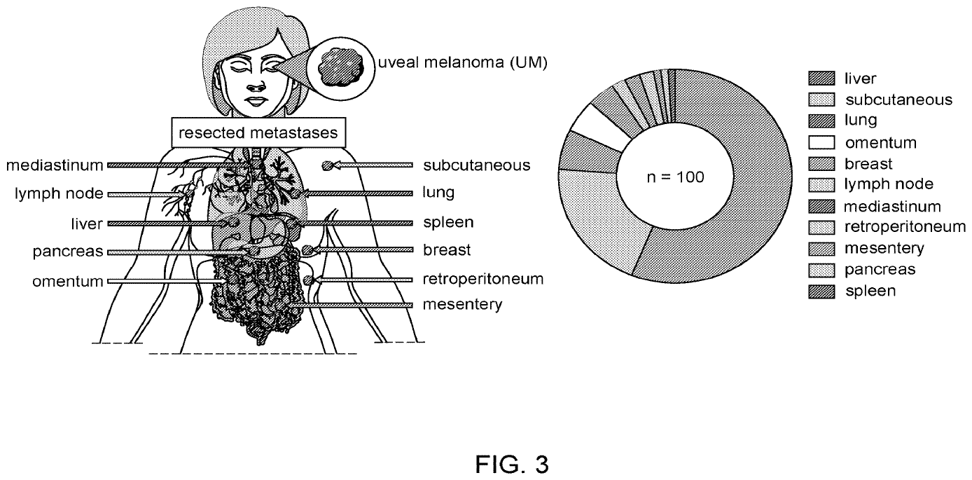

Uveal melanoma metastases are poorly immunogenic cancers. 100 uveal melanoma metastases were surgically resected from 84 patients, with 12 patients undergoing multiple tumor harvests. The locations of the harvested metastases are shown in FIG. 3. The median age of the patients was 56 years, with 52% of the patients being female and 48% of the patients being male. The median tumor mutation burden (TMB) was 0.64 mutations/Megabase. 46/84 of the patients were subjected to previous checkpoint blockade. None of the patients were checkpoint blockade responders.

Harvested tissues were obtained from tumors using a core-like biopsy to obtain samples that were 2-3 mm in size. Samples were snap frozen until analysis was performed. Harvested tissues also underwent TIL expansion and immunologic interrogation using current methods.

Total RNAseq was performed on harvested tissues for both coding and noncoding RNA. Roughly 40 million reads per sample were obtained, and counts were obtained using a custom and static bioinformatics pipeline.

### Principal Components Analysis (PCA)

To determine which principal components (PCs) contribute meaningful variance, a 10% variance threshold was used. Only those that contributed ≥10% of variance were deemed relevant (PC1, PC2, PC3).

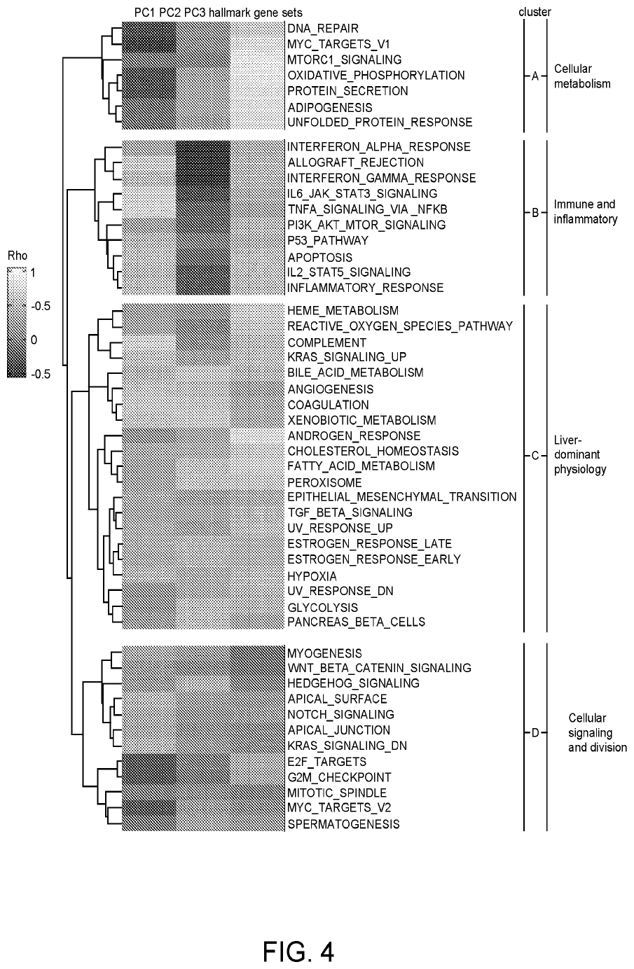

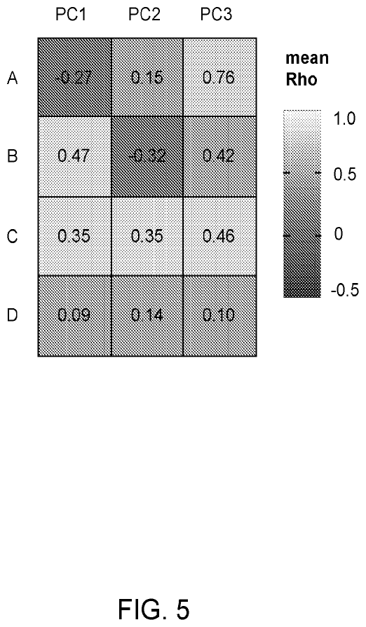

To correlate PCs with established biologic signatures, enrichment scores were calculated for samples of a MSigDB Hallmark geneset collection using singscore, and the calculated scores were correlated with PC sample loadings. PC correlations with hallmark genesets are shown in FIG. 4. Functional characterization of clusters was performed based upon the contained gene sets in each cluster (FIG. 4). Specific PC-cluster relationships were examined using mean rho (FIG. 5). Cluster A (cellular metabolism) was strongly correlated with PC3 (mean rho=+0.76) but also weakly correlated with the negative aspect of PC1 (mean rho=−0.27) (FIG. 5). Cluster B (immune and inflammatory signaling) was exclusively correlated with the negative aspect of PC2 (rho=−0.32) (FIG. 5). Clusters C and D were not found to independently correlate with any of the three PCs (FIG. 5).

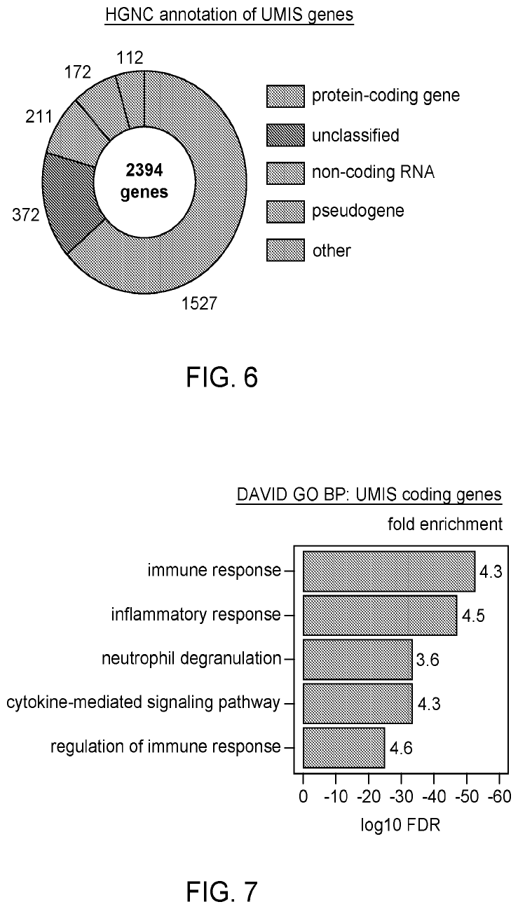

To interrogate PC2, the PC of interest given its unique correlation with immune gene sets, gene loadings for PC2 were interrogated, and contributions of genes to positive or negative aspects of PC3 were identified. 2394 genes of interest were identified that correlated with PC2 in the same direction as hallmark immune genesets. Gene type and functional annotation showed coding and noncoding genes (FIG. 6) and immune-related functions (FIG. 7).

The PC2 immune genelist (n=2394) was subjected to the singscore method using transcripts per million (TPM) input to obtain a rank-based approach resulting in a single score per sample with an enrichment score ranging from 0 to 1.

### Validation and Application of TILScore

### Multihistology Analysis

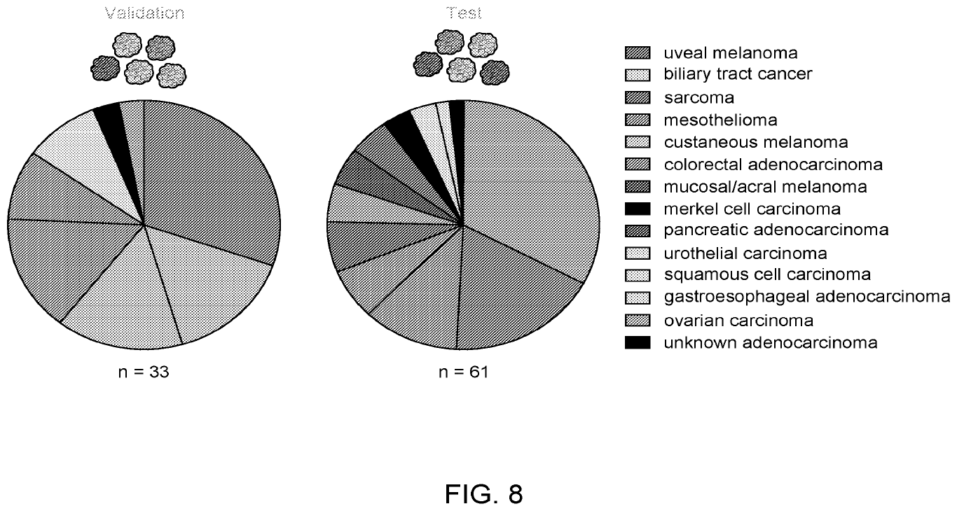

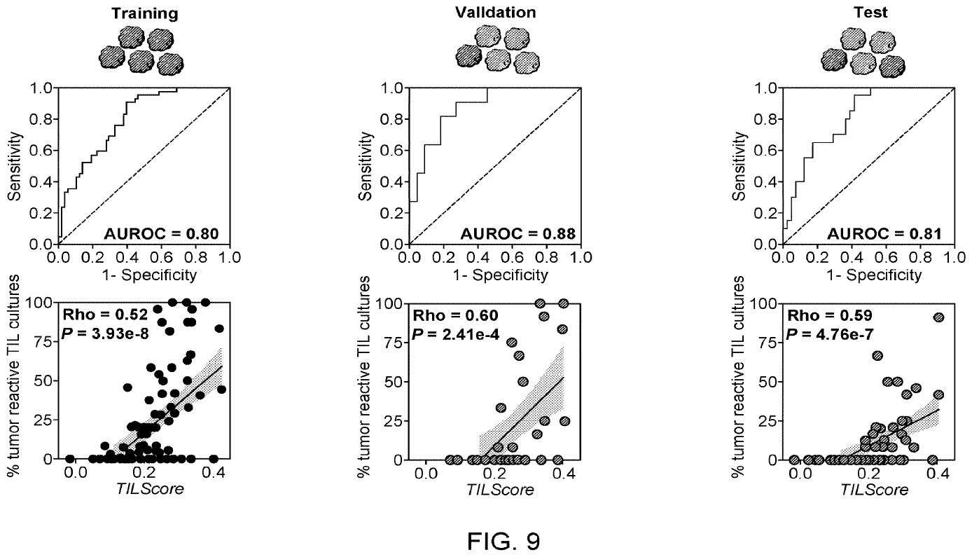

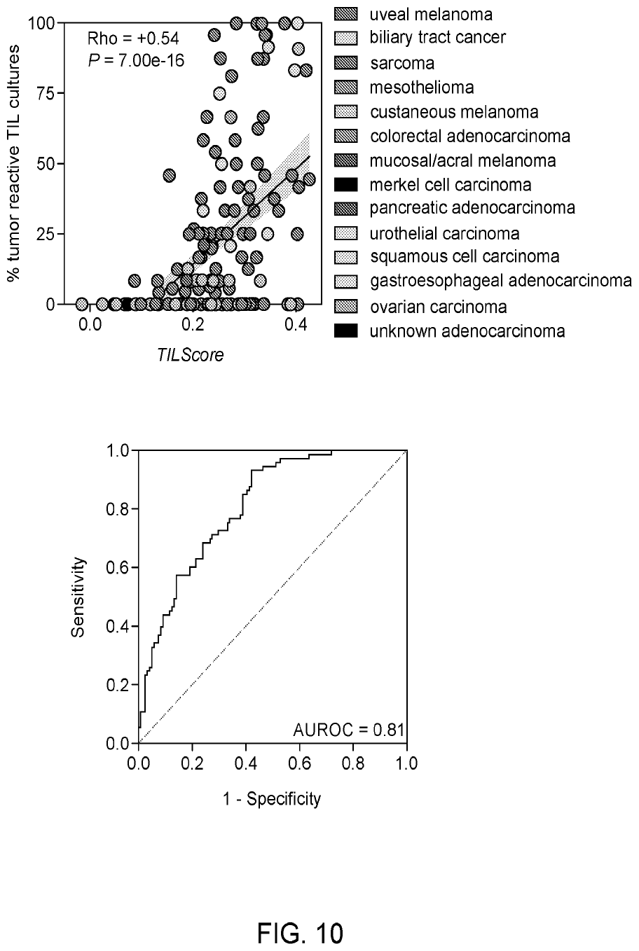

To determine whether the genelist of 2394 can predict TIL reactivity in other poorly immunogenic solid tumors, 94 samples of multiple different solid tumor types were obtained (FIG. 8). Immunologic studies and RNAseq plus TILScore were applied to these samples. Genes were ranked by strength of correlation with immunologic TIL reactivity in the training (100 uveal melanoma) cohort. The list of 2394 was then sequentially shaved from weakest correlated gene to strongest and each gene set tested for ability to predict TIL reactivity in the training and validation cohorts. The geneset of 265 (Table 1) was selected based upon its combined strongest predictive value in the validation cohort and named TILScore. TILScore correlated with and successfully classified TIL reactivity in the training, validation, and independent test cohorts (FIG. 9). A pan-cancer cohort was generated by combining the metastatic uveal melanoma samples and the multihistology samples (FIG. 10), and TILScore analysis was run. The TILScore correlated with and successfully classified TIL reactivity in the combined pan-cancer cohort (FIG. 10).

### TILScore can Guide Harvest Site Selection

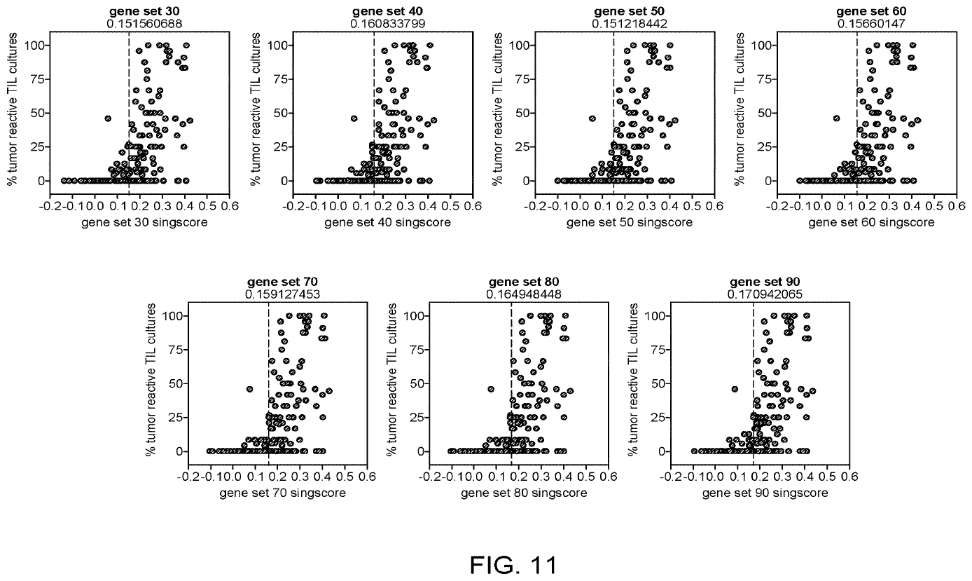

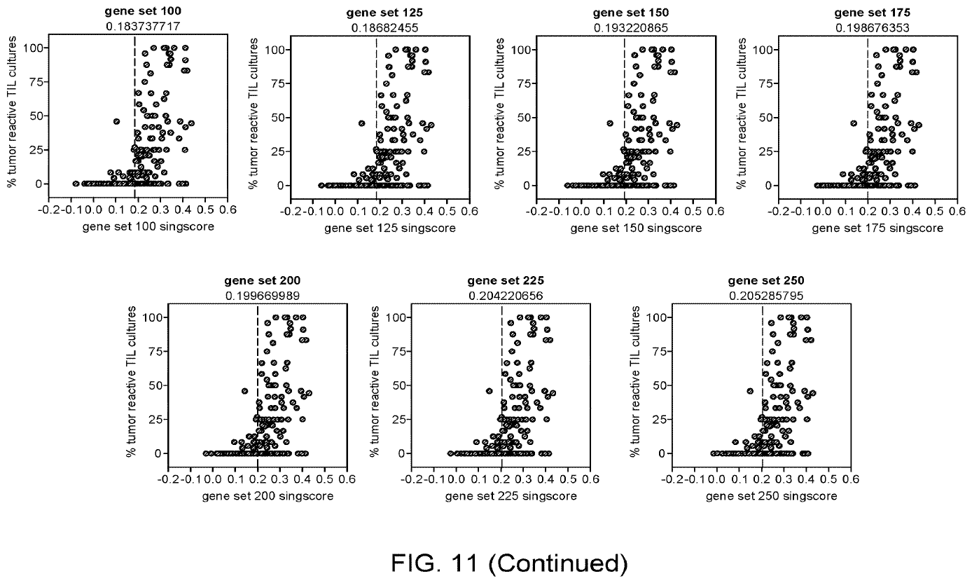

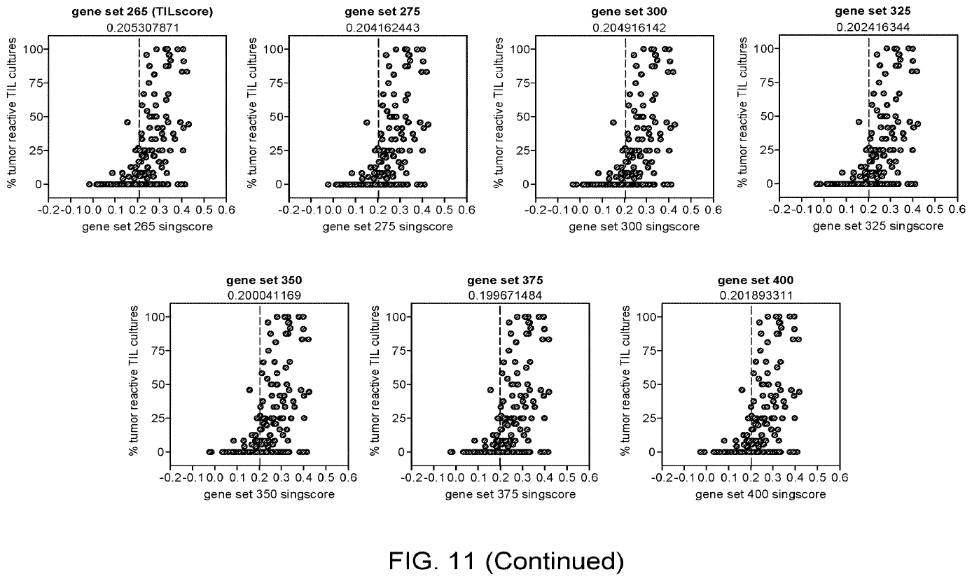

To determine whether the TILScore can preoperatively identify metastases sites and guide harvest site selection, a TILScore reactivity probability calculator was generated using the following formula:

$$TILScore probability ( from TRAIN 100 + VALID 33 )$$

$$Logistic regression from \geq 10 \% tumor - reactive TIL cultures$$

$$p ( x ) = σ ( t ) = \frac{1}{1 + {e}^{- t}}$$

$$where :$$

$$x = TILScore$$

$$t = {B}_{0} + {B}_{1} x$$

$${B}_{0} = - 4.514455$$

$${B}_{1} = 16.637663$$

$$mean TILscore = 0.2359621$$

$$standard deviation = 0.08796414$$

where P is probability, e is Euler's number, Bₒ is −4.514455, and B₁ₓ is 16.637663. Different thresholds for 25% probability of generating ≥10% tumor reactive TIL cultures were calculated based on different numbers of gene sets used (including the selected TILScore of 265) (FIG. 11).

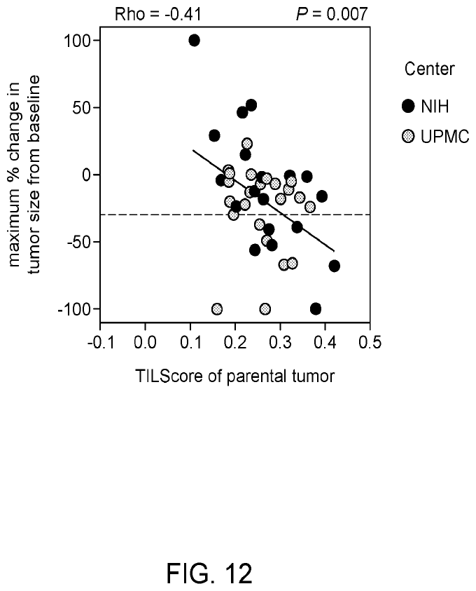

To determine whether the TILScore can predict TIL clinical efficacy after ACT, RNAseq and TILScore was applied to uveal melanoma samples obtained from 42 patients undergoing TIL therapy. TILScore predicted RECIST response in these patients undergoing TIL therapy (FIG. 12). These results demonstrate that TILScore predicted clinical response to ACT of TIL based upon a score generated from a pre-treatment core or core-like biopsy and transcriptomic profiling.

### TILScore Predicts TIL Reactivity in Poorly Immunogenic Cancers

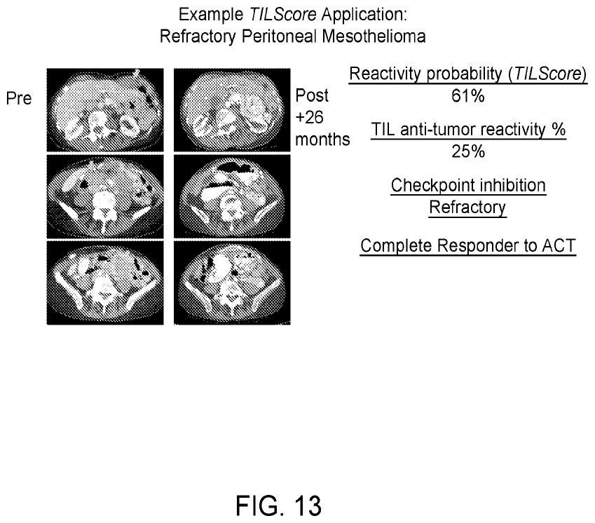

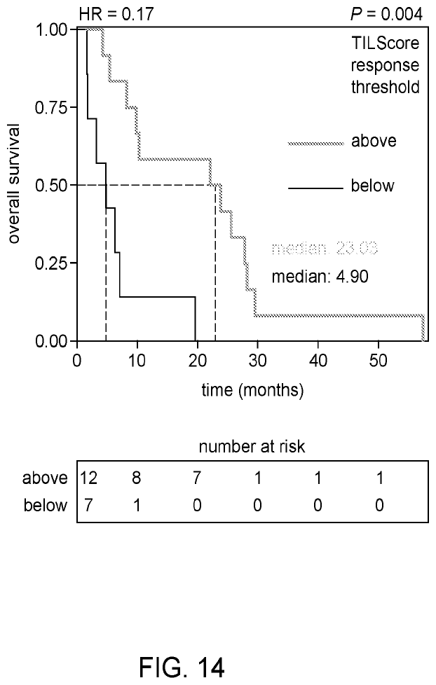

To determine whether the TILScore can be applied clinically to other poorly immunogenic cancers, TILScores were calculated in from a sample from a refractory peritoneal mesothelioma patient, a 60-year-old female with metastatic malignant peritoneal mesothelioma. The patient was previously treated with: 1) pemetrexed and cisplatin, 2) CRS/HIPEC with mitomycin-C (adjuvant pemetrexed and carboplatin), 3) right thoracotomy, debulking of right chest (adjuvant pemetrexed and carboplatin), 4) radiotherapy (5000 cGy to paraesophageal nodes), 5) palliative small bowel bypass with end colostomy, 6) anti-PD-1 therapy, and/or 7) anti-CTLA-4 plus anti-PD-1 therapy, and the patient experienced recurrence or progression on all these therapies. The TILScore was found to be 0.297, and the probability of TIL reactivity was found to be 60.7% for this patient. The TILScore predicted TIL reactivity and ACT response (FIG. 13) in this patient with refractory peritoneal mesothelioma.

### Example 2: Top 265 Nucleic Acids Used to Generate a TILScore

Nucleic acids were ranked in an order of contribution to effectively determining whether or not a cancer is likely to be responsive to an ACT such as a TIL therapy (Table 1).

TABLE 1

| Nucleic Acid Name | Rank |
|---|---|
| LINC01215 | 1 |
| HLA-DOB | 2 |
| TNFRSF9 | 3 |
| IRAG2 | 4 |
| LINC01619 | 5 |
| CTLA4 | 6 |
| CD69 | 7 |
| IL10RA | 8 |
| FCRL3 | 9 |
| RN7SL368P | 10 |
| PTPN22 | 11 |
| AIM2 | 12 |
| STAT4 | 13 |
| PRDM8 | 14 |
| TIGIT | 15 |
| GBP5 | 16 |
| KLRK1 | 17 |
| GPR84 | 18 |
| SLA | 19 |
| GFI1 | 20 |
| VAV3 | 21 |
| CD247 | 22 |
| CLEC4E | 23 |
| LINC02446 | 24 |
| CYRIA | 25 |
| BTLA | 26 |
| TNFSF8 | 27 |
| SYTL3 | 28 |
| PTPRC | 29 |
| APOBEC3A | 30 |
| HSH2D | 31 |
| ZC3H12D | 32 |
| CRTAM | 33 |
| KLRD1 | 34 |
| TRGC2 | 35 |
| TNFAIP3 | 36 |
| ZNF831 | 37 |
| PATL2 | 38 |
| AC015911.7 | 39 |
| DTHD1 | 40 |
| IL2RG | 41 |
| IPCEF1 | 42 |
| SH2D1A | 43 |
| PTPN7 | 44 |
| IRF1 | 45 |
| RASSF5 | 46 |
| SCML4 | 47 |
| LIPN | 48 |
| RASGRP1 | 49 |
| TCL1A | 50 |
| TAP1 | 51 |
| SLA2 | 52 |
| HCAR3 | 53 |
| ITK | 54 |
| P2RY10 | 55 |
| IL7R | 56 |
| CD38 | 57 |
| ERMN | 58 |
| CYTIP | 59 |
| NT5C3AP2 | 60 |
| CORO1A | 61 |
| ELAPOR1 | 62 |
| RHOH | 63 |
| ARRDC5 | 64 |
| RPL32P1 | 65 |
| CST7 | 66 |
| ZBTB32 | 67 |
| CD7 | 68 |
| ICOS | 69 |
| ARHGAP15 | 70 |
| BIRC3 | 71 |
| AL359962.2 | 72 |
| PDCD1 | 73 |
| C11orf21 | 74 |
| HLA-F | 75 |
| NCR1 | 76 |
| TAGAP | 77 |
| HLA-B | 78 |
| GPR18 | 79 |
| DAPP1 | 80 |
| CCR7 | 81 |
| SNX20 | 82 |
| TRBC1 | 83 |
| TRAF3IP3 | 84 |
| TRBC2 | 85 |
| ARHGAP9 | 86 |
| IL21R | 87 |
| FASLG | 88 |
| AC004847.1 | 89 |
| NLRC5 | 90 |
| LCP2 | 91 |
| TESPA1 | 92 |
| PIM2 | 93 |
| HLA-C | 94 |
| HCST | 95 |
| PRDM1 | 96 |
| CHI3L2 | 97 |
| SP140 | 98 |
| SRGN | 99 |
| CD2 | 100 |
| FLT3 | 101 |
| NOD2 | 102 |
| IL12RB1 | 103 |
| NCF1B | 104 |
| WDFY4 | 105 |
| HLA-E | 106 |
| THEMIS | 107 |
| CLEC12A | 108 |
| ETV7 | 109 |
| KLHDC7B | 110 |
| MAP3K8 | 111 |
| TBC1D10C | 112 |
| GZMB | 113 |
| RGL4 | 114 |
| SLAMF1 | 115 |
| BISPR | 116 |
| TNIP3 | 117 |
| TBX21 | 118 |
| TAP2 | 119 |
| TNFRSF1B | 120 |
| JAML | 121 |
| LINC01934 | 122 |
| CD96 | 123 |
| SAMSN1 | 124 |
| IKZF1 | 125 |
| CD8A | 126 |
| GBP2 | 127 |
| TRAT1 | 128 |
| CXCR4 | 129 |
| SPDYA | 130 |
| CASS4 | 131 |
| TRG-AS1 | 132 |
| GPR132 | 133 |
| DOK2 | 134 |
| SLAMF6 | 135 |
| LCP1 | 136 |
| RUNX3 | 137 |
| CCDC194 | 138 |
| NUAK2 | 139 |
| HCLS1 | 140 |
| PSMB9 | 141 |
| LINC00299 | 142 |
| SGPP2 | 143 |
| SLAMF7 | 144 |
| TREML2 | 145 |
| NCKAP1L | 146 |
| PRF1 | 147 |
| SPOCK2 | 148 |
| MIR222HG | 149 |
| NLRP3 | 150 |
| AC144831.1 | 151 |
| SPN | 152 |
| PIK3R5 | 153 |
| NIBAN3 | 154 |
| SIRPG | 155 |
| MIR22HG | 156 |
| CD53 | 157 |
| AC079316.2 | 158 |
| LY9 | 159 |
| AC090617.5 | 160 |
| CTSW | 161 |
| DGKA | 162 |
| ARHGAP30 | 163 |
| WAKMAR2 | 164 |
| IRF8 | 165 |
| ZBED2 | 166 |
| CD8B | 167 |
| SIDT1 | 168 |
| STK17B | 169 |
| PARP15 | 170 |
| ITGAL | 171 |
| DOCK2 | 172 |
| TRAC | 173 |
| SP140L | 174 |
| HCP5 | 175 |
| PCED1B-AS1 | 176 |
| SHISAL2A | 177 |
| CCR5 | 178 |
| IKZF3 | 179 |
| INPP5D | 180 |
| PLAC8 | 181 |
| JAK3 | 182 |
| SLFN12L | 183 |
| AOAH | 184 |
| CD48 | 185 |
| CD28 | 186 |
| ARAP2 | 187 |
| PLEK | 188 |
| AC079316.1 | 189 |
| SECTM1 | 190 |
| CCR4 | 191 |
| GBP3 | 192 |
| LINC00528 | 193 |
| CD5 | 194 |
| TOX | 195 |
| MEFV | 196 |
| ST8SIA4 | 197 |
| AC243960.1 | 198 |
| GPR15 | 199 |
| GBP1 | 200 |
| NFKBIZ | 201 |
| RN7SL600P | 202 |
| IL15 | 203 |
| MYO1G | 204 |
| RAC2 | 205 |
| LILRB2 | 206 |
| RASAL3 | 207 |
| IFITM1 | 208 |
| ITGB2-AS1 | 209 |
| C16orf54 | 210 |
| TSPAN32 | 211 |
| ACAP1 | 212 |
| PIK3CG | 213 |
| CCL5 | 214 |
| C1orf162 | 215 |
| GPR171 | 216 |
| NKG7 | 217 |
| P2RX1 | 218 |
| EPSTI1 | 219 |
| GNA15 | 220 |
| CCL4 | 221 |
| IL2RB | 222 |
| AC008764.10 | 223 |
| HEATR9 | 224 |
| VAV1 | 225 |
| LAX1 | 226 |
| CD27 | 227 |
| SELL | 228 |
| LTB | 229 |
| RUFY4 | 230 |
| AL353795.3 | 231 |
| IL1B | 232 |
| TMC8 | 233 |
| RGS1 | 234 |
| CD6 | 235 |
| ADAM19 | 236 |
| AL671883.2 | 237 |
| DRAIC | 238 |
| IFI30 | 239 |
| STAT1 | 240 |
| CSF2RB | 241 |
| HCAR2 | 242 |
| CRYBG1 | 243 |
| IL18RAP | 244 |
| RELT | 245 |
| TTC24 | 246 |
| NLRP1 | 247 |
| PTPRE | 248 |
| ANTXRLP1 | 249 |
| NLRC3 | 250 |
| CD300E | 251 |
| TOMM20P2 | 252 |
| AC004687.1 | 253 |
| CD3D | 254 |
| RUBCNL | 255 |
| AC104695.3 | 256 |
| AC025580.2 | 257 |
| SH2D2A | 258 |
| LCK | 259 |
| AC015911.3 | 260 |
| AC090559.1 | 261 |
| RNU6-1010P | 262 |
| DENND2D | 263 |
| RPS16P5 | 264 |
| LINC01436 | 265 |

### Example 3: Gene ID Numbers for Top 265 Nucleic Acids for Humans Used to Generate a TILScore

The HUGO Gene Nomenclature Committee Gene ID (HGNC_ID) number for each nucleic acid listed in Table 1 for humans is as set forth in Table 2.

TABLE 2

| Nucleic Acid | | Nucleic Acid | | Nucleic Acid | |
|---|---|---|---|---|---|
| Name | HGNC_ID | Name | HGNC_ID | Name | HGNC_ID |
| LINC01215 | 49651 | NLRC5 | 29933 | IKZF3 | 13178 |
| HLA-DOB | 4937 | LCP2 | 6529 | INPP5D | 6079 |
| TNFRSF9 | 11924 | TESPA1 | 29109 | PLAC8 | 19254 |
| IRAG2 | 6690 | PIM2 | 8987 | JAK3 | 6193 |
| LINC01619 | 27409 | HLA-C | 4933 | SLFN12L | 33920 |
| CTLA4 | 2505 | HCST | 16977 | AOAH | 548 |
| CD69 | 1694 | PRDM1 | 9346 | CD48 | 1683 |
| IL10RA | 5964 | CHI3L2 | 1933 | CD28 | 1653 |
| FCRL3 | 18506 | SP140 | 17133 | ARAP2 | 16924 |
| RN7SL368P | 46384 | SRGN | 9361 | PLEK | 9070 |
| PTPN22 | 9652 | CD2 | 1639 | AC079316.1 | NA |
| AIM2 | 357 | FLT3 | 3765 | SECTM1 | 10707 |
| STAT4 | 11365 | NOD2 | 5331 | CCR4 | 1605 |
| PRDM8 | 13993 | IL12RB1 | 5971 | GBP3 | 4184 |
| TIGIT | 26838 | NCF1B | 32522 | LINC00528 | 26875 |
| GBP5 | 19895 | WDFY4 | 29323 | CD5 | 1685 |
| KLRK1 | 18788 | HLA-E | 4962 | TOX | 18988 |
| GPR84 | 4535 | THEMIS | 21569 | MEFV | 6998 |
| SLA | 10902 | CLEC12A | 31713 | ST8SIA4 | 10871 |
| GFI1 | 4237 | ETV7 | 18160 | AC243960.1 | NA |
| VAV3 | 12659 | KLHDC7B | 25145 | GPR15 | 4469 |
| CD247 | 1677 | MAP3K8 | 6860 | GBP1 | 4182 |
| CLEC4E | 14555 | TBC1D10C | 24702 | NFKBIZ | 29805 |
| LINC02446 | 53378 | GZMB | 4709 | RN7SL600P | 46616 |
| CYRIA | 25373 | RGL4 | 31911 | IL15 | 5977 |
| BTLA | 21087 | SLAMF1 | 10903 | MYO1G | 13880 |
| TNFSF8 | 11938 | BISPR | 51290 | RAC2 | 9802 |
| SYTL3 | 15587 | TNIP3 | 19315 | LILRB2 | 6606 |
| PTPRC | 9666 | TBX21 | 11599 | RASAL3 | 26129 |
| APOBEC3A | 17343 | TAP2 | 44 | IFITM1 | 5412 |
| HSH2D | 24920 | TNFRSF1B | 11917 | ITGB2-AS1 | 44304 |
| ZC3H12D | 21175 | JAML | 19084 | C16orf54 | 26649 |
| CRTAM | 24313 | LINC01934 | 52757 | TSPAN32 | 13410 |
| KLRD1 | 6378 | CD96 | 16892 | ACAP1 | 16467 |
| TRGC2 | 12276 | SAMSN1 | 10528 | PIK3CG | 8978 |
| TNFAIP3 | 11896 | IKZF1 | 13176 | CCL5 | 10632 |
| ZNF831 | 16167 | CD8A | 1706 | C1orf162 | 28344 |
| PATL2 | 33630 | GBP2 | 4183 | GPR171 | 30057 |
| AC015911.7 | NA | TRAT1 | 30698 | NKG7 | 7830 |
| DTHD1 | 37261 | CXCR4 | 2561 | P2RX1 | 8533 |
| IL2RG | 6010 | SPDYA | 30613 | EPSTI1 | 16465 |
| IPCEF1 | 21204 | CASS4 | 15878 | GNA15 | 4383 |
| SH2D1A | 10820 | TRG-AS1 | 48974 | CCL4 | 10630 |
| PTPN7 | 9659 | GPR132 | 17482 | IL2RB | 6009 |
| IRF1 | 6116 | DOK2 | 2991 | AC008764.10 | NA |
| RASSF5 | 17609 | SLAMF6 | 21392 | HEATR9 | 26548 |
| SCML4 | 21397 | LCP1 | 6528 | VAV1 | 12657 |
| LIPN | 23452 | RUNX3 | 10473 | LAX1 | 26005 |
| RASGRP1 | 9878 | CCDC194 | 53438 | CD27 | 11922 |
| TCL1A | 11648 | NUAK2 | 29558 | SELL | 10720 |
| TAP1 | 43 | HCLS1 | 4844 | LTB | 6711 |
| SLA2 | 17329 | PSMB9 | 9546 | RUFY4 | 24804 |
| HCAR3 | 16824 | LINC00299 | 27940 | AL353795.3 | NA |
| ITK | 6171 | SGPP2 | 19953 | IL1B | 5992 |
| P2RY10 | 19906 | SLAMF7 | 21394 | TMC8 | 20474 |
| IL7R | 6024 | TREML2 | 21092 | RGS1 | 9991 |
| CD38 | 1667 | NCKAP1L | 4862 | CD6 | 1691 |
| ERMN | 29208 | PRF1 | 9360 | ADAM19 | 197 |
| CYTIP | 9506 | SPOCK2 | 13564 | AL671883.2 | NA |
| NT5C3AP2 | 54578 | MIR222HG | 49555 | DRAIC | 27082 |
| CORO1A | 2252 | NLRP3 | 16400 | IFI30 | 5398 |
| ELAPOR1 | 29618 | AC144831.1 | NA | STAT1 | 11362 |
| RHOH | 686 | SPN | 11249 | CSF2RB | 2436 |
| ARRDC5 | 31407 | PIK3R5 | 30035 | HCAR2 | 24827 |
| RPL32P1 | 10339 | NIBAN3 | 24130 | CRYBG1 | 356 |
| CST7 | 2479 | SIRPG | 15757 | IL18RAP | 5989 |
| ZBTB32 | 16763 | MIR22HG | 28219 | RELT | 13764 |
| CD7 | 1695 | CD53 | 1686 | TTC24 | 32348 |
| ICOS | 5351 | AC079316.2 | NA | NLRP1 | 14374 |
| ARHGAP15 | 21030 | LY9 | 6730 | PTPRE | 9669 |
| BIRC3 | 591 | AC090617.5 | NA | ANTXRLP1 | 45004 |
| AL359962.2 | NA | CTSW | 2546 | NLRC3 | 29889 |
| PDCD1 | 8760 | DGKA | 2849 | CD300E | 28874 |
| C11orf21 | 13231 | ARHGAP30 | 27414 | TOMM20P2 | 50519 |
| HLA-F | 4963 | WAKMAR2 | 53754 | AC004687.1 | NA |
| NCR1 | 6731 | IRF8 | 5358 | CD3D | 1673 |
| TAGAP | 15669 | ZBED2 | 20710 | RUBCNL | 20420 |
| HLA-B | 4932 | CD8B | 1707 | AC104695.3 | NA |
| GPR18 | 4472 | SIDT1 | 25967 | AC025580.2 | NA |
| DAPP1 | 16500 | STK17B | 11396 | SH2D2A | 10821 |
| CCR7 | 1608 | PARP15 | 26876 | LCK | 6524 |
| SNX20 | 30390 | ITGAL | 6148 | AC015911.3 | NA |
| TRBC1 | 12156 | DOCK2 | 2988 | AC090559.1 | NA |
| TRAF3IP3 | 30766 | TRAC | 12029 | RNU6-1010P | 47973 |
| TRBC2 | 12157 | SP140L | 25105 | DENND2D | 26192 |
| ARHGAP9 | 14130 | HCP5 | 21659 | RPS16P5 | 36183 |
| IL21R | 6006 | PCED1B-AS1 | 44166 | LINC01436 | 50754 |
| FASLG | 11936 | SHISAL2A | 28757 | | |
| AC004847.1 | NA | CCR5 | 1606 | | |

### Example 4: Evaluation of Gene Sets of Different Sizes

To determine the performance of candidate gene sets, a standardized clinically relevant success: futility cutoff for selected gene sets was evaluated. The success: futility cutoff was defined as the ability to generate ~10% tumor reactive TIL cultures from a single metastasis per the gold standard laboratory assay. The graphs in FIG. 11 display candidate gene set enrichment scores using singscores (x-axis) versus % tumor reactive TIL cultures (y-axis) generated from 194 individual metastases.

Success: futility cutoffs were derived from training+validation cohorts of metastases (n=133) and denote the singscore enrichment values equating to 25% probability of yielding significant tumor reactive TIL cultures (~10%). Dashed lines and values in FIG. 11 represent success: futility cutoffs overlaid onto the total cohort of 194 individual metastases.

### Example 5: Assessing Cancer for TIL Reactivity

A tumor tissue sample is obtained from a human having cancer (e.g., metastatic cancer). The obtained sample is assessed to calculate a TILScore of the sample based on a relative rank of at least 80 percent (e.g., at least 90 percent, or 100 percent) of the 30 nucleic acids listed in Table 1 as having rank #1 to rank #30.

If the sample has a TILScore of at least 0.15 (e.g., at least 0.15, at least 0.16, at least 0.17, at least 0.18, at least 0.19, at least 0.2, at least 0.3, at least 0.4, at least 0.5, or at least 0.6), then the cancer is classified as being responsive to an ACT (e.g., a TIL therapy).

If the sample has a TILScore of less than 0.15, then the cancer can be classified as not being responsive to an ACT (e.g., a TIL therapy).

### Example 6: Preparing a Treatment for a Human Having Cancer

A tumor tissue sample is obtained from a human having cancer (e.g., metastatic cancer). The obtained sample is assessed to calculate a TILScore of the sample based on a relative rank of at least 80 percent (e.g., at least 90 percent, or 100 percent) of the 30 nucleic acids listed in Table 1 as having rank #1 to rank #30.

If the sample has a TILScore of at least 0.15 (e.g., at least 0.15, at least 0.16, at least 0.17, at least 0.18, at least 0.19, at least 0.2, at least 0.3, at least 0.4, at least 0.5, or at least 0.6), then TILs obtained from that human are expanded ex vivo. Once expanded, the TILs are formulated into a composition for administration to the human to treat the human's cancer.

If the sample has a TILScore of less than 0.15, then one can avoid or stop the preparation of TILs for that human, and other cancer treatments can be discussed.

### Example 7: Treating Cancer

A tumor tissue sample is obtained from a human having cancer (e.g., metastatic cancer). The obtained sample is assessed to calculate a TILScore of the sample based on a relative rank of at least 80 percent (e.g., at least 90 percent, or 100 percent) of the 30 nucleic acids listed in Table 1 as having rank #1 to rank #30.

If the sample has a TILScore of at least 0.15 (e.g., at least 0.15, at least 0.16, at least 0.17, at least 0.18, at least 0.19, at least 0.2, at least 0.3, at least 0.4, at least 0.5, or at least 0.6), then TILs obtained from that human are expanded ex vivo. Once expanded, the TILs are formulated into a composition for administration to the human to treat the human's cancer, and the formulated composition is administered to the human. Once administered to the human, the TILs can reduce the number of cancer cells present in the human.

### Example 8: Treating Cancer

A tumor tissue sample is obtained from a human having cancer (e.g., metastatic cancer). The obtained sample is assessed to calculate a TILScore of the sample based on a relative rank of at least 80 percent (e.g., at least 90 percent, or 100 percent) of the 30 nucleic acids listed in Table 1 as having rank #1 to rank #30.

If the sample has a TILScore of less than 0.15, then the human can be administered one or more (e.g., one, two, three, four, five, or more) alternative cancer treatments (e.g., one or more cancer treatments that are not an ACT such as a TIL therapy). The alternative cancer treatment can reduce the number of cancer cells present in the human.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for identifying a mammal having cancer likely to respond to an adoptive cell therapy (ACT), wherein said method comprises:
(a) determining that a sample obtained from said mammal and comprising cancer cells has a tumor infiltrating lymphocyte score (TILScore) of at least 0.15, wherein said TILScore is calculated based on a relative rank of at least 30 nucleic acids listed in Table 1; and
(b) classifying said cancer as being likely to respond to said ACT.

2. The method of claim 1, wherein said mammal is a human.

3. (canceled)

4. The method of claim 1, wherein said cancer comprises a solid tumor.

5-14. (canceled)

15. A method for selecting a treatment for a mammal having cancer, wherein said method comprises:
(a) determining that a sample obtained from said mammal and comprising cancer cells has a TILScore of at least 0.15, wherein said TILScore is calculated based on a relative rank of at least 30 nucleic acids listed in Table 1; and
(b) selecting an ACT as a treatment of cancer for said mammal.

16. The method of claim 15, wherein said mammal is a human.

17. (canceled)

18. The method of claim 15, wherein said cancer comprises a solid tumor.

19-30. (canceled)

31. A method for preparing a treatment for a mammal having cancer, wherein said method comprises:
(a) determining that a sample obtained from said mammal and comprising cancer cells has a TILScore of at least 0.15, wherein said TILScore is calculated based on a relative rank of at least 30 nucleic acids listed in Table 1; and
(b) expanding TILs obtained from said mammal ex vivo to obtain expanded TILs for administration to said mammal.

32. The method of claim 31, wherein said mammal is a human.

33. The method of claim 31, wherein said cancer comprises a solid tumor.

34-39. (canceled)

40. The method of claim 31, wherein said method further comprises administering at least a portion of said expanded TILs to said mammal.

41. A method for preparing a cancer treatment, wherein said method comprises expanding TILs obtained from a mammal identified as having a TILScore of at least 0.15 to form a cell population for administration to said mammal to treat cancer, wherein said TILScore is calculated based on a relative rank of at least 30 nucleic acids listed in Table 1.

42. The method of claim 41, wherein said mammal is a human.

43. The method of claim 41, wherein said cancer comprises a solid tumor.

44-49. (canceled)

50. The method of claim 41, wherein said method further comprises administering at least a portion of said cell population to said mammal.

51. A method for treating a mammal having cancer, wherein said method comprises:
(a) determining that a sample obtained from said mammal and comprising cancer cells has a TILScore of at least 0.15, wherein said TILScore is calculated based on a relative rank of at least 30 nucleic acids listed in Table 1; and
(b) administering an ACT to said mammal.

52. The method of claim 51, wherein said mammal is a human.

53. (canceled)

54. The method of claim 51, wherein said cancer comprises a solid tumor.

55-60. (canceled)

61. A method for treating cancer, wherein said method comprises administering an ACT to a mammal identified as having a TILScore of at least 0.15, wherein said TILScore is determined from a sample obtained from said mammal and comprising cancer cells, and wherein said TILScore is calculated based on a relative rank of at least 30 nucleic acids listed in Table 1.

62. The method of claim 61, wherein said mammal is a human.

63. (canceled)

64. The method of claim 61, wherein said cancer comprises a solid tumor.

65-84. (canceled)
